(19) United States
(12) Patent Application Publication
(10) Pub. No.: US 2007/0292485 A1
(43) Pub. Date: Dec. 20, 2007

(54) POLYOL ESTER COMPOUNDS

(75) Inventors: Stephen J. Lorbert, St. Louis, MO (US); Thomas Frederick Blackburn, Buffalo, MO (US); Charles S. Schasteen, St. Louis, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: Novus International Inc., St. Louis, MO (US)

(21) Appl. No.: 11/764,394

(22) Filed: Jun. 18, 2007

Related U.S. Application Data
(60) Provisional application No. 60/804,956, filed on Jun. 16, 2006.

(51) Int. Cl. A61K 31/4178; A61K 31/405; A61K 31/22; C07D 403/02; C07D 209/02

(52) U.S. Cl. 424/442; 548/325.5; 554/85; 548/454; 548/465; 554/104

(57) ABSTRACT

Disclosed are polyol esters comprising a polyol having at least one hydroxyl group esterified to at least one compound having a carboxyl group. Methods of making the polyol ester compounds are also disclosed. The invention also relates to feed compositions comprising polyol ester compounds.

### CROSS-REFERENCE TO RELATED APPLICATIONS

This application claims priority to U.S. Provisional Application Ser. No. 60/804,956 filed on Jun. 16, 2006, which is hereby incorporated by reference in its entirety.

### FIELD OF THE INVENTION

The present invention generally relates to polyol esters comprising a polyol having at least one hydroxyl group esterified to at least one compound having a carboxyl group.

### BACKGROUND OF THE INVENTION

There is a growing demand for nutritional supplements or liquid food supplements that provide nutrients, vitamins, and/or minerals to humans and animals. Such supplements have traditionally been given to infants, the elderly, or severely ill patients to provide life-saving nutrition. Nutritional supplements may also be used by athletes to boost strength and performance or by ordinary persons with hectic lifestyles to provide a balanced diet. Furthermore, they may be given to companion animals to meet their nutritional needs or to agricultural animals to promote growth and health.

Nutritional supplements may contain essential amino acids, i.e., those amino acids that cannot be synthesized by humans or animals. Lysine, methionine, phenyalanine, and tryptophan are examples of essential amino acids frequently added to foods. A popular source of supplemental methionine for animal diets is the methionine analog, 2-hydroxy-4-(methylthio)butanoic acid (HMTBA, sold under the trade name ALIMET® by Novus International, Inc., St. Louis, Mo.). HMTBA is absorbed by the cells of the small and large intestine by passive diffusion and is rapidly converted to methionine. Another advantage of HMTBA is that high concentrations may be given to animals, whereas high levels of methionine are toxic. One problem associated with the addition of free amino acids to a food supplement, however, is that the amino acid may be degraded in the harsh environment of the stomach.

Nutritional supplements may also contain a quick energy source in the form of fatty acids or triglycerides (glycerol esters of fatty acids) rather than glucose or another form of sugar. Short chain (C₂-C₆) fatty acids (e.g., acetate, propionic acid, butanoic acid, lactic acid) are typically generated in the large intestine by microbial fermentation of non-digestible starches or soluble fiber. Short chain fatty acids are readily absorbed and oxidized for energy or used to generate ATP. The addition of short chain fatty acids or short chain triglycerides to a nutritional supplement enables these fatty acids to be absorbed earlier in the intestinal tract. Medium chain (C₈-C₁₂) triglycerides are regularly added to infant formulas because breast milk is highly enriched with these molecules. Medium chain triglycerides are digested and absorbed much more quickly than long chain triglycerides, and thus provide a quick source of energy. Both short chain and medium chain fatty acids acidify the intestine, thereby, providing antimicrobial activity by restricting the growth and activity of less beneficial bacterial species. A problem associated with adding free fatty acids to a food supplement is the need to administer equivalent amounts of cations.

While it is well established that nutrition supplements may contain amino acids and/or fatty acids (i.e., carboxylic acids), there is a need for a mechanism to deliver sufficient quantities of these nutrients in an intact state to the intestine for ready absorption. The amino acid and/or carboxylic acid should be linked to the delivery system by a covalent bond that is stable in the stomach but hydrolyzed by enzymes (i.e., lipases and esterases) in the intestine. The delivery system should also be able of carrying more than one molecule at a time and a combination of different types of molecules.

### SUMMARY OF THE INVENTION

The present invention provides polyol di-ester or tri-ester compounds in which the polyol is esterified to at least one substituted carboxylic acid having Formula (I), as defined herein. The polyol ester may be further esterified to one or more carboxylic acids of two to twenty-two carbons, one or more substituted carboxylic acids of two to twenty-two carbons, one or more amino acids, or combinations thereof. In one exemplary alternative of this embodiment, the polyol is glycerol.

In yet another aspect, the invention provides processes for the preparation of polyol ester and glycerol ester compounds.

The present invention also provides feed compositions comprising a polyol ester compound or a mixture of polyol ester compounds. The feed composition may be a dry supplement or an aqueous formation.

### DETAILED DESCRIPTION OF THE PREFERRED EMBODIMENTS

(I) Polyol Ester Compounds

The invention provides polyol ester compounds. Generally speaking, a compound of the invention may comprise a polyol esterified to at least one compound containing a carboxyl group. Processes suitable for making polyol esters of the invention are detailed herein (e.g., in section (II) and the examples) or are otherwise generally known in the art.

(a) Polyols

Suitable polyols typically have at least one accessible hydroxyl group. In this context, the term “accessible” means the hydroxyl group of the polyol is capable of forming an ester bond with a compound containing a carboxyl group. More typically, the polyol may have three or more hydroxyl groups. A suitable polyol having three hydroxyl groups is glycerol. In other embodiments, the polyol may be a sugar alcohol having four to six hydroxyl groups. Examples of suitable sugar alcohols include erythritol, xylitol, sorbitol, maltitol and mannitol. In an alternative embodiment, the polyol may be an oligosaccharide or polysaccharide having at least one accessible hydroxyl group. Insulin is an example of a suitable oligosaccharide.

(b) Carboxyl Containing Compounds

A variety of suitable compounds having at least one carboxyl group may be used to form polyol esters. The compound is generally an organic acid having a carboxyl group. Typically, the compound will be a carboxylic acid or a substituted carboxylic acid.

The carboxylic acid compound may be a monocarboxylic acid having a straight chain or it may be branched; it may be saturated or unsaturated. In one embodiment, the carboxylic acid may contain from about two to about twenty-five carbon atoms. In another embodiment, the carboxylic acid may have from about three to about twenty-two carbon atoms. In a further embodiment, the carboxylic acid may contain from about three to about twelve carbon atoms. In yet another embodiment, the carboxylic acid may contain from about eight to about twelve carbon atoms. In still another embodiment, the carboxylic acid may contain from about two to about six carbon atoms. By way of non limiting example, the carboxylic acid may be a saturated aliphatic compound selected from the group consisting of propanoic acid, butanoic acid, pentaenoic acid, caproic or hexanoic acid, heptanoic acid, caprylic or octanoic acid, nonanoic acid, capric or decanoic acid, undecanoic acid, lauric or dodecanoic acid, tridecanoic acid, myristic or tetradecanoic acid, pentadecanoic acid, palmitic or hexadecanoic acid, margaric or heptadecanoic acid, stearic or octadecanoic acid, nonadecanoic acid, arachidic or eicosanoic acid, and behenic or docosanoic acid. Alternatively, the carboxylic acid may be an unsaturated aliphatic compound selected from the group consisting of sorbic acid, a hexanoic acid with two double bonds (6:2), myristoleic acid (i.e., a C₁₄ acid with one double bond (14:1)), palmitoleic acid (16:1), oleic acid (18:1), linoleic acid (18:2), linolenic (18:3), gadoleic acid (20:1), and arachidonic acid (20:4).

In another embodiment, the carboxylic acid compound may be a substituted carboxylic acid. A substituted carboxylic acid generally has the same features as those detailed above for carboxylic acids, but the hydrocarbyl chain has been modified such that it is branched, is part of a ring structure, or contains some other substitution. In one embodiment, the substituted carboxylic acid may contain one or more additional carboxyl groups. Saturated dicarboxylic acids include malonic acid, succinic acid, glutaric acid, and adipic acid, and unsaturated dicarboxylic acids include maleic acid and fumaric acid. In another embodiment, the substituted carboxylic acid may contain one or more hydroxyl groups. A substituted carboxylic acid with a hydroxyl group on the alpha carbon, i.e., the carbon adjacent to the carboxyl carbon, is generally called a α-hydroxy carboxylic acid. Examples of suitable α-hydroxy carboxylic acids include glycolic acid, lactic acid, malic acid, and tartaric acid. In an alternate embodiment, the substituted carboxylic acid may contain one or more carbonyl groups. In yet another embodiment, the substituted carboxylic acid may contain an amino group on the alpha carbon, i.e., is an α-amino acid. In one embodiment, the α-amino acid may be one of the twenty standard amino acids or derivatives thereof. In another embodiment, the α-amino acid may be an essential α-amino acid selected from the group consisting of arginine, histidine, isoleucine, leucine, lysine, methionine, phenylalanine, threonine, tryptophan, and valine.

In yet another embodiment, the substituted carboxylic acid may be a compound having Formula (I):

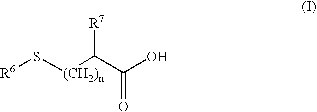

wherein:
n is an integer from 0 to 2;
R⁶ is an alkyl group having from one to four carbon atoms;
R⁷ is selected from the group consisting of hydroxyl, amino, and —OCOR⁸ or —NHCOR⁸; and
R⁸ is an organic acid derivative.

In an exemplary embodiment for compounds having Formula (I), R⁶ is methyl or ethyl; R⁷ is hydroxyl or amino; and n is 0 to 2.

Salts of compounds having Formula (I) are also suitable for certain embodiments. Representative salts of the compound of Formula (I) include the ammonium, magnesium, calcium, lithium, sodium, potassium, selenium, iron, copper, and zinc salts. In a preferred embodiment, the compound of Formula (I) is in the form of the calcium salt. Representative amides include methylamide, dimethylamide, ethylmethylamide, butylamide, dibutylamide, butylmethylamide, alkyl ester of N-acyl methionates (e.g., alkyl N-acetyl methionates. Representative esters include the methyl, ethyl, n-propyl, isopropyl, butyl esters, namely n-butyl, sec-butyl, isobutyl, and t-butyl esters, pentyl esters and hexyl esters, especially n-pentyl, isopentyl, n-hexyl and isohexyl esters.

In various preferred embodiments, the compound of Formula (I) is 2-hydroxy-4-(methylthio)butanoic acid (HMTBA) or a salt, amide or ester thereof, such as any of those detailed above. In still more preferred embodiments, the compound of Formula (I) is HMTBA.

In other embodiments, exemplary compounds having Formula (I) may be selected from the group consisting of:
1-hydroxy-l-(methylthio)acetic acid;
1-hydroxy-l-(ethylthio)acetic acid;
1-hydroxy-l-(propylthio)acetic acid;
1-hydroxy-l-(butylthio)acetic acid;
1-amino-l-(methylthio)acetic acid;
1-amino-l-(ethylthio)acetic acid;
1-amino-l-(propylthio)acetic acid;
1-amino-l-(butylthio)acetic acid;
1-carboxy-l-(methylthio)acetic acid;
1-acetyloxy-l-(methylthio)acetic acid;
1-propionyloxy-l-(methylthio)acetic acid;
1-butyryloxy-l-(methylthio)acetic acid;
1-benzoyloxy-l-(methylthio)acetic acid;
1-lactoyloxy-l-(methylthio)acetic acid;
1-[2-carboxy-2-(hydroxy)propionyloxy]-1-(methylthio)acetic acid;
1-[2-carboxy-l-(hydroxy)propionyloxy]-1-(methylthio)acetic acid;
1-[2-carboxy-1,2-(dihydroxy)propionyloxy]-1-(methylthio)acetic acid;
1-[hydroxy(phenyl)acetyl]oxy-1-(methylthio)acetic acid;
1-[2,3-dicarboxy-2-(hydroxy)butyryloxy]-1-(methylthio)acetic acid;
1-[2-carboxy-1-carboxymethyl-1-(hydroxy)propionyloxy]-1-(methylthio)acetic acid;
1-(3-carboxyacryloyl)oxy-1-(methylthio)acetic acid;
1-(2,4-pentadienoyloxy)-1-(methylthio)acetic acid;
1-(2-carboxypropionyloxy)-1-(methylthio)acetic acid;
1-[(4-carboxy)amyloxy]-1-(methylthio)acetic acid;
1-glycoloyloxy-l-(methylthio)acetic acid;
1-glutaroyloxy-l-(methylthio)acetic acid;
1-formylamino-l-(methylthio)acetic acid;
1-acetylamino-l-(methylthio)acetic acid;
1-propionylamino-l-(methylthio)acetic acid;
1-butyrylamino-l-(methylthio)acetic acid;
1-benzoylamino-l-(methylthio)acetic acid;
1-lactoylamino-l-(methylthio)acetic acid;
1-[2-carboxy-2-(hydroxy)propionylamino]-1-(methylthio)acetic acid;
1-[2-carboxy-1-(hydroxy)propionylamino]-1-(methylthio)acetic acid;
1-[2-carboxy-1,2-(dihydroxy)propionylamino]-l-(methylthio)acetic acid;
1-[hydroxy(phenyl)acetyl]amino-1-(methylthio) acetic acid;
1-[2,3-dicarboxy-2-(hydroxy)butyrylamino]-1-(methylthio) acetic acid;
1-[2-carboxy-l-carboxymethyl-1-(hydroxy)propionylamino]-1-(methylthio)acetic acid;
1-(3-carboxyacryloyl)amino-1-(methylthio)acetic acid;
1-(2,4-pentadienoylamino)-1-(methylthio)acetic acid;
1-(2-carboxypropionylamino)-1-(methylthio)acetic acid;
1-[(4-carboxy)amylamino]-1-(methylthio)acetic acid;
1-glycoloylamino-1-(methylthio)acetic acid;
1-glutaroylamino-1-(methylthio)acetic acid;
1-carboxy-(ethylthio)acetic acid;
1-acetyloxy-(ethylthio)acetic acid;
1-propionyloxy-(ethylthio)acetic acid;
1-butyryloxy-(ethylthio)acetic acid;
1-benzoyloxy-(ethylthio)acetic acid;
1-lactoyloxy-(ethylthio)acetic acid;
1-[2-carboxy-2-(hydroxy)propionyloxy]-(ethylthio)acetic acid;
1-[2-carboxy-l-(hydroxy)propionyloxy]-(ethylthio)acetic acid;
1-[2-carboxy-1,2-(dihydroxy)propionyloxy]-(ethylthio)acetic acid;
1-[hydroxy(phenyl)acetyl]oxy-(ethylthio)acetic acid;
1-[2,3-dicarboxy-2-(hydroxy)butyryloxy]-(ethylthio)acetic acid;
1-[2-carboxy-1-carboxymethyl-1-(hydroxy)propionyloxy]-(ethylthio)acetic acid;
1-(3-carboxyacryloyl)oxy-(ethylthio)acetic acid;
1-(2,4-pentadienoyloxy)-(ethylthio)acetic acid;
1-(2-carboxypropionyloxy)-(ethylthio)acetic acid;
1-[(4-carboxy)amyloxy]-(ethylthio)acetic acid;
1-glycoloyloxy-(ethylthio)acetic acid;
1-glutaroyloxy-(ethylthio)acetic acid;
1-formylamino-(ethylthio)acetic acid;
1-acetylamino-(ethylthio)acetic acid;
1-propionylamino-(ethylthio)acetic acid;
1-butyrylamino-(ethylthio)acetic acid;
1-benzoylamino-(ethylthio)acetic acid;
1-lactoylamino-(ethylthio)acetic acid;
1-[2-carboxy-2-(hydroxy)propionylamino]-(ethylthio)acetic acid;
1-[2-carboxy-1-(hydroxy)propionylamino]-(ethylthio)acetic acid;
1-[2-carboxy-1,2-(dihydroxy)propionylamino]-(ethylthio)acetic acid;
1-[hydroxy(phenyl)acetyl]amino-(ethylthio)acetic acid;
1-[2,3-dicarboxy-2-(hydroxy)butyrylamino]-(ethylthio)acetic acid;
1-[2-carboxy-1-carboxymethyl-1-(hydroxy)propionylamino]-(ethylthio)acetic acid;
1-(3-carboxyacryloyl)amino-(ethylthio)acetic acid;
1-(2,4-pentadienoylamino)-(ethylthio)acetic acid;
1-(2-carboxypropionylamino)-(ethylthio)acetic acid;
1-[(4-carboxy)amylamino]-(ethylthio)acetic acid;
1-glycoloylamino-(ethylthio)acetic acid;
1-glutaroylamino-(ethylthio)acetic acid;
1-carboxy-(propylthio)acetic acid;
1-acetyloxy-(propylthio)acetic acid;
1-propionyloxy-(propylthio)acetic acid;
1-butyryloxy-(propylthio)acetic acid;
1-benzoyloxy-(propylthio)acetic acid;
1-lactoyloxy-(propylthio)acetic acid;
1-[2-carboxy-2-(hydroxy)propionyloxy]-(propylthio)acetic acid;
1-[2-carboxy-l-(hydroxy)propionyloxy]-(propylthio)acetic acid;
1-[2-carboxy-1,2-(dihydroxy)propionyloxy]-(propylthio)acetic acid;
1-[hydroxy(phenyl)acetyl]oxy-(propylthio)acetic acid;
1-[2,3-dicarboxy-2-(hydroxy)butyryloxy]-(propylthio)acetic acid;
1-[2-carboxy-1-carboxymethyl-1-(hydroxy)propionyloxy]-(propylthio)acetic acid;
1-(3-carboxyacryloyl)oxy-(propylthio)acetic acid;
1-(2,4-pentadienoyloxy)-(propylthio)acetic acid;
1-(2-carboxypropionyloxy)-(propylthio)acetic acid;
1-[(4-carboxy)amyloxy]-(propylthio)acetic acid;
1-glycoloyloxy-(propylthio)acetic acid;
1-glutaroyloxy-(propylthio)acetic acid;
1-formylamino-(propylthio)acetic acid;
1-acetylamino-(propylthio)acetic acid;
1-propionylamino-(propylthio)acetic acid;
1-butyrylamino-(propylthio)acetic acid;
1-benzoylamino-(propylthio)acetic acid;
1-lactoylamino-(propylthio)acetic acid;
1-[2-carboxy-2-(hydroxy)propionylamino]-(propylthio)acetic acid;
1-[2-carboxy-l-(hydroxy)propionylamino]-(propylthio)acetic acid;
1-[2-carboxy-1,2-(dihydroxy)propionylamino]-(propylthio)acetic acid;
1-[hydroxy(phenyl)acetyl]amino-(propylthio)acetic acid;
1-[2,3-dicarboxy-2-(hydroxy)butyrylamino]-(propylthio)acetic acid;
1-[2-carboxy-l-carboxymethyl-1-(hydroxy)propionylamino]-(propylthio)acetic acid;
1-(3-carboxyacryloyl)amino-(propylthio)acetic acid;
1-(2,4-pentadienoylamino)-(propylthio)acetic acid;
1-(2-carboxypropionylamino)-(propylthio)acetic acid;
1-[(4-carboxy)amylamino]-(propylthio)acetic acid;
1-glycoloylamino-(propylthio)acetic acid;
1-glutaroylamino-(propylthio)acetic acid;
1-carboxy-(butylthio)acetic acid;
1-acetyloxy-(butylthio)acetic acid;
1-propionyloxy-(butylthio)acetic acid;
1-butyryloxy-(butylthio)acetic acid;
1-benzoyloxy-(butylthio)acetic acid;
1-lactoyloxy-(butylthio)acetic acid;
1-[2-carboxy-2-(hydroxy)propionyloxy]-(butylthio)acetic acid;
1-[2-carboxy-1-(hydroxy)propionyloxy]-(butylthio)acetic acid;
1-[2-carboxy-1,2-(dihydroxy)propionyloxy]-(butylthio)acetic acid;
1-[hydroxy (phenyl)acetyl]oxy-(butylthio)acetic acid;

1-[2,3-dicarboxy-2-(hydroxy)butyryloxy]-(butylthio)acetic acid;
1-[2-carboxy-l-carboxymethyl-1-(hydroxy)propionyloxy]-(butylthio)acetic acid;
1-(3-carboxyacryloyl)oxy-(butylthio)acetic acid;
1-(2,4-pentadienoyloxy)-(butylthio)acetic acid;
1-(2-carboxypropionyloxy)-(butylthio)acetic acid;
1-[(4-carboxy)amyloxy]-(butylthio)acetic acid;
1-glycoloyloxy-(butylthio)acetic acid;
1-glutaroyloxy-(butylthio)acetic acid;
1-formylamino-(butylthio)acetic acid;
1-acetylamino-(butylthio)acetic acid;
1-propionylamino-(butylthio)acetic acid;
1-butyrylamino-(butylthio)acetic acid;
1-benzoylamino-(butylthio)acetic acid;
1-lactoylamino-(butylthio)acetic acid;
1-[2-carboxy-2-(hydroxy)propionylamino]-(butylthio)acetic acid;
1-[2-carboxy-l-(hydroxy)propionylamino]-(butylthio)acetic acid;
1-[2-carboxy-1,2-(dihydroxy)propionylamino]-(butylthio)acetic acid;
1-[hydroxy(phenyl)acetyl]amino-(butylthio) acetic acid;
1-[2,3-dicarboxy-2-(hydroxy)butyrylamino]-(butylthio)acetic acid;
1-[2-carboxy-1-carboxymethyl-1-(hydroxy)propionylamino]-(butylthio)acetic acid;
1-(3-carboxyacryloyl)amino-(butylthio)acetic acid;
1-(2,4-pentadienoylamino)-(butylthio)acetic acid;
1-(2-carboxypropionylamino)-(butylthio)acetic acid;
1-[(4-carboxy)amylamino]-(butylthio)acetic acid;
1-glycoloylamino-(butylthio)acetic acid;
1-glutaroylamino-(butylthio)acetic acid;
2-hydroxy-3-(methylthio)propanoic acid;
2-hydroxy-3-(ethylthio)propanoic acid;
2-hydroxy-3-(propylthio)propanoic acid;
2-hydroxy-3-(butylthio)propanoic acid;
2-amino-3-(methylthio)propanoic acid;
2-amino-3-(ethylthio)propanoic acid;

2-amino-3-(propylthio)propanoic acid;
2-amino-3-(butylthio)propanoic acid;
2-carboxy-3-(methylthio)propanoic acid;
2-acetyloxy-3-(methylthio)propanoic acid;
2-propionyloxy-3-(methylthio)propanoic acid;
2-butyryloxy-3-(methylthio)propanoic acid;
2-benzoyloxy-3-(methylthio)propanoic acid;
2-lactoyloxy-3-(methylthio)propanoic acid;
2-[2-carboxy-2-(hydroxy)propionyloxy]-3-(methylthio)propanoic acid;
2-[2-carboxy-1-(hydroxy)propionyloxy]-3-(methylthio)propanoic acid;
2-[2-carboxy-1,2-(dihydroxy)propionyloxy]-3-(methylthio)propanoic acid;
2-[hydroxy(phenyl)acetyl]oxy-3-(methylthio)propanoic acid;
2-[2,3-dicarboxy-2-(hydroxy)butyryloxy]-3-(methylthio)propanoic acid;
2-[2-carboxy-1-carboxymethyl-1-(hydroxy)propionyloxy]-3-(methylthio)propanoic acid;
2-(3-carboxyacryloyl)oxy-3-(methylthio)propanoic acid;
2-(2,4-pentadienoyloxy)-3-(methylthio)propanoic acid;
2-(2-carboxypropionyloxy)-3-(methylthio)propanoic acid;
2-[(4-carboxy)amyloxy]-3-(methylthio)propanoic acid;
2-glycoloyloxy-3-(methylthio)propanoic acid;
2-glutaroyloxy-3-(methylthio)propanoic acid;
2-formylamino-3-(methylthio)propanoic acid;
2-acetylamino-3-(methylthio)propanoic acid;
2-propionylamino-3-(methylthio)propanoic acid;
2-butyrylamino-3-(methylthio)propanoic acid;
2-benzoylamino-3-(methylthio)propanoic acid;
2-lactoylamino-3-(methylthio)propanoic acid;
2-[2-carboxy-2-(hydroxy)propionylamino]-3-(methylthio)propanoic acid;
2-[2-carboxy-1-(hydroxy)propionylamino]-3-(methylthio)propanoic acid;
2-[2-carboxy-1,2-(dihydroxy)propionylamino]-3-(methylthio)propanoic acid;
2-[hydroxy(phenyl)acetyl]amino-3-(methylthio)propanoic acid;
2-[2,3-dicarboxy-2-(hydroxy)butyrylamino]-3-(methylthio)propanoic acid;
2-[2-carboxy-l-carboxymethyl-l-(hydroxy)propionylamino]-3-(methylthio)propanoic acid;
2-(3-carboxyacryloyl)amino-3-(methylthio)propanoic acid;
2-(2,4-pentadienoylamino)-3-(methylthio)propanoic acid;
2-(2-carboxypropionylamino)-3-(methylthio)propanoic acid;
2-[(4-carboxy)amylamino]-3-(methylthio)propanoic acid;
2-glycoloylamino-3-(methylthio)propanoic acid;
2-glutaroylamino-3-(methylthio)propanoic acid;
2-carboxy-3-(ethylthio)propanoic acid;
2-acetyloxy-3-(ethylthio)propanoic acid;
2-propionyloxy-3-(ethylthio)propanoic acid;
2-butyryloxy-3-(ethylthio)propanoic acid;
2-benzoyloxy-3-(ethylthio)propanoic acid;
2-lactoyloxy-3-(ethylthio)propanoic acid;
2-[2-carboxy-2-(hydroxy)propionyloxy]-3-(ethylthio)propanoic acid;
2-[2-carboxy-l-(hydroxy)propionyloxy]-3-(ethylthio)propanoic acid;
2-[2-carboxy-1,2-(dihydroxy)propionyloxy]-3-(ethylthio)propanoic acid;
2-[hydroxy (phenyl)acetyl]oxy-3-(ethylthio)propanoic acid;
2-[2,3-dicarboxy-2-(hydroxy)butyryloxy]-3-(ethylthio)propanoic acid;
2-[2-carboxy-1-carboxymethyl-1-(hydroxy)propionyloxy]-3-(ethylthio)propanoic acid;
2-(3-carboxyacryloyl)oxy-3-(ethylthio)propanoic acid;
2-(2,4-pentadienoyloxy)-3-(ethylthio)propanoic acid;
2-(2-carboxypropionyloxy)-3-(ethylthio)propanoic acid;
2-[(4-carboxy)amyloxy]-3-(ethylthio)propanoic acid;
2-glycoloyloxy-3-(ethylthio)propanoic acid;
2-glutaroyloxy-3-(ethylthio)propanoic acid;
2-formylamino-3-(ethylthio)propanoic acid;
2-acetylamino-3-(ethylthio)propanoic acid;
2-propionylamino-3-(ethylthio)propanoic acid;
2-butyrylamino-3-(ethylthio)propanoic acid;
2-benzoylamino-3-(ethylthio)propanoic acid;
2-lactoylamino-3-(ethylthio)propanoic acid;
2-[2-carboxy-2-(hydroxy)propionylamino]-3-(ethylthio)propanoic acid;
2-[2-carboxy-l-(hydroxy)propionylamino]-3-(ethylthio)propanoic acid;
2-[2-carboxy-1,2-(dihydroxy)propionylamino]-3-(ethylthio)propanoic acid;
2-[hydroxy(phenyl)acetyl]amino-3-(ethylthio)propanoic acid;
2-[2,3-dicarboxy-2-(hydroxy)butyrylamino]-3-(ethylthio)propanoic acid;
2-[2-carboxy-l-carboxymethyl-l-(hydroxy)propionylamino]-3-(ethylthio)propanoic acid;
2-(3-carboxyacryloyl)amino-3-(ethylthio)propanoic acid;
2-(2,4-pentadienoylamino)-3-(ethylthio)propanoic acid;
2-(2-carboxypropionylamino)-3-(ethylthio)propanoic acid;
2-[(4-carboxy)amylamino]-3-(ethylthio)propanoic acid;
2-glycoloylamino-3-(ethylthio)propanoic acid;
2-glutaroylamino-3-(ethylthio)propanoic acid;
2-carboxy-3-(propylthio)propanoic acid;
2-acetyloxy-3-(propylthio)propanoic acid;
2-propionyloxy-3-(propylthio)propanoic acid;
2-butyryloxy-3-(propylthio)propanoic acid;
2-benzoyloxy-3-(propylthio)propanoic acid;
2-lactoyloxy-3-(propylthio)propanoic acid;
2-[2-carboxy-2-(hydroxy)propionyloxy]-3-(propylthio)propanoic acid;
2-[2-carboxy-1-(hydroxy)propionyloxy]-3-(propylthio)propanoic acid;
2-[2-carboxy-1,2-(dihydroxy)propionyloxy]-3-(propylthio)propanoic acid;
2-[hydroxy(phenyl)acetyl]oxy-3-(propylthio)propanoic acid;
2-[2,3-dicarboxy-2-(hydroxy)butyryloxy]-3-(propylthio)propanoic acid;
2-[2-carboxy-1-carboxymethyl-1-(hydroxy)propionyloxy]-3-(propylthio)propanoic acid;
2-(3-carboxyacryloyl)oxy-3-(propylthio)propanoic acid;
2-(2,4-pentadienoyloxy)-3-(propylthio)propanoic acid;
2-(2-carboxypropionyloxy)-3-(propylthio)propanoic acid;
2-[(4-carboxy)amyloxy]-3-(propylthio)propanoic acid;
2-glycoloyloxy-3-(propylthio)propanoic acid;
2-glutaroyloxy-3-(propylthio)propanoic acid;
2-formylamino-3-(propylthio)propanoic acid;
2-acetylamino-3-(propylthio)propanoic acid;
2-propionylamino-3-(propylthio)propanoic acid;
2-butyrylamino-3-(propylthio)propanoic acid;
2-benzoylamino-3-(propylthio)propanoic acid;
2-lactoylamino-3-(propylthio)propanoic acid;
2-[2-carboxy-2-(hydroxy)propionylamino]-3-(propylthio)propanoic acid;
2-[2-carboxy-1-(hydroxy)propionylamino]-3-(propylthio)propanoic acid;
2-[2-carboxy-1,2-(dihydroxy)propionylamino]-3-(propylthio)propanoic acid;
2-[hydroxy(phenyl)acetyl]amino-3-(propylthio)propanoic acid;
2-[2,3-dicarboxy-2-(hydroxy)butyrylamino]-3-(propylthio)propanoic acid;
2-[2-carboxy-l-carboxymethyl-l-(hydroxy)propionylamino]-3-(propylthio)propanoic acid;
2-(3-carboxyacryloyl)amino-3-(propylthio)propanoic acid;
2-(2,4-pentadienoylamino)-3-(propylthio)propanoic acid;
2-(2-carboxypropionylamino)-3-(propylthio)propanoic acid;
2-[(4-carboxy)amylamino]-3-(propylthio)propanoic acid;
2-glycoloylamino-3-(propylthio)propanoic acid;
2-glutaroylamino-3-(propylthio)propanoic acid;
2-carboxy-3-(butylthio)propanoic acid;
2-acetyloxy-3-(butylthio)propanoic acid;
2-propionyloxy-3-(butylthio)propanoic acid;
2-butyryloxy-3-(butylthio)propanoic acid;
2-benzoyloxy-3-(butylthio)propanoic acid;
2-lactoyloxy-3-(butylthio)propanoic acid;
2-[2-carboxy-2-(hydroxy)propionyloxy]-3-(butylthio)propanoic acid;
2-[2-carboxy-1-(hydroxy)propionyloxy]-3-(butylthio)propanoic acid;
2-[2-carboxy-1,2-(dihydroxy)propionyloxy]-3-(butylthio)propanoic acid;
2-[hydroxy(phenyl)acetyl]oxy-3-(butylthio)propanoic acid;
2-[2,3-dicarboxy-2-(hydroxy)butyryloxy]-3-(butylthio)propanoic acid;
2-[2-carboxy-1-carboxymethyl-1-(hydroxy)propionyloxy]-3-(butylthio)propanoic acid;
2-(3-carboxyacryloyl)oxy-3-(butylthio)propanoic acid;
2-(2,4-pentadienoyloxy)-3-(butylthio)propanoic acid;
2-(2-carboxypropionyloxy)-3-(butylthio)propanoic acid;

2-[(4-carboxy)amyloxy]-3-(butylthio)propanoic 2-glycoloyloxy-3-(butylthio)propanoic acid;
2-glutaroyloxy-3-(butylthio)propanoic acid;
2-formylamino-3-(butylthio)propanoic acid;
2-acetylamino-3-(butylthio)propanoic acid;
2-propionylamino-3-(butylthio)propanoic acid;
2-butyrylamino-3-(butylthio)propanoic acid;
2-benzoylamino-3-(butylthio)propanoic acid;
2-lactoylamino-3-(butylthio)propanoic acid;
2-[2-carboxy-2-(hydroxy)propionylamino]-3-(butylthio)propanoic acid;
2-[2-carboxy-1-(hydroxy)propionylamino]-3-(butylthio)propanoic acid;
2-[2-carboxy-1,2-(dihydroxy)propionylamino]-3-(butylthio)propanoic acid;
2-[hydroxy(phenyl)acetyl]amino-3-(butylthio)propanoic acid;
2-[2,3-dicarboxy-2-(hydroxy)butyrylamino]-3-(butylthio)propanoic acid;
2-[2-carboxy-1-carboxymethyl-1-(hydroxy)propionylamino]-3-(butylthio)propanoic acid;
2-(3-carboxyacryloyl)amino-3-(butylthio)propanoic acid;
2-(2,4-pentadienoylamino)-3-(butylthio)propanoic acid;
2-(2-carboxypropionylamino)-3-(butylthio)propanoic acid;
2-[(4-carboxy)amylamino]-3-(butylthio)propanoic acid;
2-glycoloylamino-3-(butylthio)propanoic acid;
2-glutaroylamino-3-(butylthio)propanoic acid;
2-hydroxy-4-(methylthio)butanoic acid;
2-hydroxy-4-(ethylthio)butanoic acid;
2-hydroxy-4-(propylthio)butanoic acid;
2-hydroxy-4-(butylthio)butanoic acid;
2-amino-4-(methylthio)butanoic acid;
2-amino-4-(ethylthio)butanoic acid;
2-amino-4-(propylthio)butanoic acid;
2-amino-4-(butylthio)butanoic acid;
2-carboxy-4-(methylthio)butanoic acid;
2-acetyloxy-4-(methylthio)butanoic acid;
2-propionyloxy-4-(methylthio)butanoic acid;

2-butyryloxy-4-(methylthio)butanoic acid;
2-benzoyloxy-4-(methylthio)butanoic acid;
2-lactoyloxy-4-(methylthio)butanoic acid;
2-[2-carboxy-2-(hydroxy)propionyloxy]-4-(methylthio)butanoic acid;
2-[2-carboxy-l-(hydroxy)propionyloxy]-4-(methylthio)butanoic acid;
2-[2-carboxy-1,2-(dihydroxy)propionyloxy]-4-(methylthio)butanoic acid;
2-[hydroxy(phenyl)acetyl]oxy-4-(methylthio)butanoic acid;
2-[2,3-dicarboxy-2-(hydroxy)butyryloxy]-4-(methylthio)butanoic acid;
2-[2-carboxy-l-carboxymethyl-1-(hydroxy)propionyloxy]-4-(methylthio)butanoic acid;
2-(3-carboxyacryloyl)oxy-4-(methylthio)butanoic acid;
2-(2,4-pentadienoyloxy)-4-(methylthio)butanoic 2-(2-carboxypropionyloxy)-4-(methylthio)butanoic acid;
2-[(4-carboxy)amyloxy]-4-(methylthio)butanoic
2-glycoloyloxy-4-(methylthio)butanoic acid;
2-glutaroyloxy-4-(methylthio)butanoic acid;
2-formylamino-4-(methylthio)butanoic acid;
2-acetylamino-4-(methylthio)butanoic acid;
2-propionylamino-4-(methylthio)butanoic acid;
2-butyrylamino-4-(methylthio)butanoic acid;
2-benzoylamino-4-(methylthio)butanoic acid;
2-lactoylamino-4-(methylthio)butanoic acid;
2-[2-carboxy-2-(hydroxy)propionylamino]-4-(methylthio)butanoic acid;
2-[2-carboxy-l-(hydroxy)propionylamino]-4-(methylthio)butanoic acid;
2-[2-carboxy-1,2-(dihydroxy)propionylamino]-4-(methylthio)butanoic acid;
2-[hydroxy(phenyl)acetyl]amino-4-(methylthio)butanoic acid;
2-[2,3-dicarboxy-2-(hydroxy)butyrylamino]-4-(methylthio)butanoic acid;
2-[2-carboxy-1-carboxymethyl-1-(hydroxy)propionylamino]-4-(methylthio)butanoic acid;
2-(3-carboxyacryloyl)amino-4-(methylthio)butanoic acid;
2-(2,4-pentadienoylamino)-4-(methylthio)butanoic acid;
2-(2-carboxypropionylamino)-4-(methylthio)butanoic acid;
2-[(4-carboxy)amylamino]-4-(methylthio)butanoic acid;

2-glycoloylamino-4-(methylthio)butanoic acid;
2-glutaroylamino-4-(methylthio)butanoic acid;
2-carboxy-4-(ethylthio)butanoic acid;
2-acetyloxy-4-(ethylthio)butanoic acid;
2-propionyloxy-4-(ethylthio)butanoic acid;
2-butyryloxy-4-(ethylthio)butanoic acid;
2-benzoyloxy-4-(ethylthio)butanoic acid;
2-lactoyloxy-4-(ethylthio)butanoic acid;
2-[2-carboxy-2-(hydroxy)propionyloxy]-4-(ethylthio)butanoic acid;
2-[2-carboxy-l-(hydroxy)propionyloxy]-4-(ethylthio)butanoic acid;
2-[2-carboxy-1,2-(dihydroxy)propionyloxy]-4-(ethylthio)butanoic acid;
2-[hydroxy(phenyl)acetyl]oxy-4-(ethylthio)butanoic acid;
2-[2,3-dicarboxy-2-(hydroxy)butyryloxy]-4-(ethylthio)butanoic acid;
2-[2-carboxy-1-carboxymethyl-1-(hydroxy)propionyloxy]-4-(ethylthio)butanoic acid;
2-(3-carboxyacryloyl)oxy-4-(ethylthio)butanoic acid;
2-(2,4-pentadienoyloxy)-4-(ethylthio)butanoic acid;
2-(2-carboxypropionyloxy)-4-(ethylthio)butanoic acid;
2-[(4-carboxy)amyloxy]-4-(ethylthio)butanoic acid;
2-glycoloyloxy-4-(ethylthio)butanoic acid;
2-glutaroyloxy-4-(ethylthio)butanoic acid;
2-formylamino-4-(ethylthio)butanoic acid;
2-acetylamino-4-(ethylthio)butanoic acid;
2-propionylamino-4-(ethylthio)butanoic acid;
2-butyrylamino-4-(ethylthio)butanoic acid;
2-benzoylamino-4-(ethylthio)butanoic acid;
2-lactoylamino-4-(ethylthio)butanoic acid;
2-[2-carboxy-2-(hydroxy)propionylamino]-4-(ethylthio)butanoic acid;
2-[2-carboxy-1-(hydroxy)propionylamino]-4-(ethylthio)butanoic acid;
2-[2-carboxy-1,2-(dihydroxy)propionylamino]-4-(ethylthio)butanoic acid;
2-[hydroxy(phenyl)acetyl]amino-4-(ethylthio)butanoic acid;
2-[2,3-dicarboxy-2-(hydroxy)butyrylamino]-4-(ethylthio)butanoic acid;
2-[2-carboxy-l-carboxymethyl-l-(hydroxy)propionylamino]-4-(ethylthio)butanoic acid;
2-(3-carboxyacryloyl)amino-4-(ethylthio)butanoic acid;
2-(2,4-pentadienoylamino)-4-(ethylthio)butanoic acid;
2-(2-carboxypropionylamino)-4-(ethylthio)butanoic acid;
2-[(4-carboxy)amylamino]-4-(ethylthio)butanoic acid;
2-glycoloylamino-4-(ethylthio)butanoic acid;
2-glutaroylamino-4-(ethylthio)butanoic acid;
2-carboxy-4-(propylthio)butanoic acid;
2-acetyloxy-4-(propylthio)butanoic acid;
2-propionyloxy-4-(propylthio)butanoic acid;
2-butyryloxy-4-(propylthio)butanoic acid;
2-benzoyloxy-4-(propylthio)butanoic acid;
2-lactoyloxy-4-(propylthio)butanoic acid;
2-[2-carboxy-2-(hydroxy)propionyloxy]-4-(propylthio)butanoic acid;
2-[2-carboxy-l-(hydroxy)propionyloxy]-4-(propylthio)butanoic acid;
2-[2-carboxy-1,2-(dihydroxy)propionyloxy]-4-(propylthio)butanoic acid;
2-[hydroxy(phenyl)acetyl]oxy-4-(propylthio)butanoic acid;
2-[2,3-dicarboxy-2-(hydroxy)butyryloxy]-4-(propylthio)butanoic acid;
2-[2-carboxy-1-carboxymethyl-1-(hydroxy)propionyloxy]-4-(propylthio)butanoic acid;
2-(3-carboxyacryloyl)oxy-4-(propylthio)butanoic acid;
2-(2,4-pentadienoyloxy)-4-(propylthio)butanoic acid;
2-(2-carboxypropionyloxy)-4-(propylthio)butanoic acid;
2-[(4-carboxy)amyloxy]-4-(propylthio) butanoic acid;
2-glycoloyloxy-4-(propylthio)butanoic acid;
2-glutaroyloxy-4-(propylthio)butanoic acid;
2-formylamino-4-(propylthio)butanoic acid;
2-acetylamino-4-(propylthio)butanoic acid;
2-propionylamino-4-(propylthio)butanoic acid;
2-butyrylamino-4-(propylthio)butanoic acid;
2-benzoylamino-4-(propylthio)butanoic acid;
2-lactoylamino-4-(propylthio)butanoic acid;
2-[2-carboxy-2-(hydroxy)propionylamino]-4-(propylthio)butanoic acid;
2-[2-carboxy-1-(hydroxy)propionylamino]-4-(propylthio)butanoic acid;
2-[2-carboxy-1,2-(dihydroxy)propionylamino]-4-(propylthio)butanoic acid;
2-[hydroxy(phenyl)acetyl]amino-4-(propylthio)butanoic acid;
2-[2,3-dicarboxy-2-(hydroxy)butyrylamino]-4-(propylthio)butanoic acid;
2-[2-carboxy-1-carboxymethyl-1-(hydroxy)propionylamino]-4-(propylthio)butanoic acid;
2-(3-carboxyacryloyl)amino-4-(propylthio)butanoic acid;
2-(2,4-pentadienoylamino)-4-(propylthio)butanoic acid;
2-(2-carboxypropionylamino)-4-(propylthio)butanoic acid;
2-[(4-carboxy)amylamino]-4-(propylthio)butanoic acid;
2-glycoloylamino-4-(propylthio)butanoic acid;
2-glutaroylamino-4-(propylthio)butanoic acid;
2-carboxy-4-(butylthio)butanoic acid;
2-acetyloxy-4-(butylthio)butanoic acid;
2-propionyloxy-4-(butylthio)butanoic acid;
2-butyryloxy-4-(butylthio)butanoic acid;
2-benzoyloxy-4-(butylthio)butanoic acid;
2-lactoyloxy-4-(butylthio)butanoic acid;
2-[2-carboxy-2-(hydroxy)propionyloxy]-4-(butylthio)butanoic acid;
2-[2-carboxy-1-(hydroxy)propionyloxy]-4-(butylthio)butanoic acid;
2-[2-carboxy-1,2-(dihydroxy)propionyloxy]-4-(butylthio)butanoic acid;
2-[hydroxy(phenyl)acetyl]oxy-4-(butylthio)butanoic acid;
2-[2,3-dicarboxy-2-(hydroxy)butyryloxy]-4-(butylthio)butanoic acid;
2-[2-carboxy-1-carboxymethyl-1-(hydroxy)propionyloxy]-4-(butylthio)butanoic acid;
2-(3-carboxyacryloyl)oxy-4-(butylthio)butanoic acid;
2-(2,4-pentadienoyloxy)-4-(butylthio)butanoic acid;
2-(2-carboxypropionyloxy)-4-(butylthio)butanoic acid;
2-[(4-carboxy)amyloxy]-4-(butylthio)butanoic acid;
2-glycoloyloxy-4-(butylthio)butanoic acid;
2-glutaroyloxy-4-(butylthio)butanoic acid;
2-formylamino-4-(butylthio)butanoic acid;
2-acetylamino-4-(butylthio)butanoic acid;
2-propionylamino-4-(butylthio)butanoic acid;
2-butyrylamino-4-(butylthio)butanoic acid;
2-benzoylamino-4-(butylthio)butanoic acid;
2-lactoylamino-4-(butylthio)butanoic acid;
2-[2-carboxy-2-(hydroxy)propionylamino]-4-(butylthio)butanoic acid;
2-[2-carboxy-1-(hydroxy)propionylamino]-4-(butylthio)butanoic acid;
2-[2-carboxy-1,2-(dihydroxy)propionylamino]-4-(butylthio)butanoic acid;
2-[hydroxy(phenyl)acetyl]amino-4-(butylthio)butanoic acid;
2-[2,3-dicarboxy-2-(hydroxy)butyrylamino]-4-(butylthio)butanoic acid;
2-[2-carboxy-1-carboxymethyl-1-(hydroxy)propionylamino]-4-(butylthio)butanoic acid;
2-(3-carboxyacryloyl)amino-4-(butylthio)butanoic acid;
2-(2,4-pentadienoylamino)-4-(butylthio)butanoic acid;
2-(2-carboxypropionylamino)-4-(butylthio)butanoic acid;
2-[(4-carboxy)amylamino]-4-(butylthio)butanoic acid;
2-glycoloylamino-4-(butylthio)butanoic acid; and
2-glutaroylamino-4-(butylthio)butanoic acid.

(c) Exemplary Polyol Ester Compounds

In one exemplary embodiment, the polyol ester is a polyol esterified to at least one substituted carboxylic acid having Formula (I) (i.e., as detailed in (I)(b)). A preferred compound having Formula (I) is 2-hydroxy-4-(methylthio)butanoic acid. The polyol may be any of the polyol compounds described in (I)(a). Preferred polyol compounds include glycerol, sorbitol, xylitol, mannitol, and insulin. In an alternative embodiment, the polyol may be esterified to at least two compounds having Formula (I). In another alternative embodiment, the polyol may be esterified to at least three compounds having Formula (I). In each of the foregoing embodiments, the polyol may be esterified to at least one or at least two carboxylic acids or substituted carboxylic acids not having Formula (I). Suitable carboxylic acids or substituted carboxylic acids include any described in (I)(b). Typically, the carboxylic acid may have from three to twenty-two carbon atoms or from three to twelve carbon atoms. Exemplary carboxylic acids include propionic acid, butanoic acid, octanoic acid, and decanoic acid. In each of the forgoing embodiments, the polyol may be esterified to an amino acid. Exemplary amino acids include essential amino acids. Suitable polyol esters according to this embodiment are detailed in Table A.

TABLE A
Polyol Ester Compounds

| Polyol | Carboxylic Acid Compound |
|---|---|
| C₃H₈O₃ (glycerol) | C₂ carboxylic acid compound |
| C₃H₈O₃ (glycerol) | C₃ carboxylic acid compound |
| C₃H₈O₃ (glycerol) | C₅ carboxylic acid compound |
| C₃H₈O₃ (glycerol) | C₆ carboxylic acid compound |
| C₃H₈O₃ (glycerol) | C₇ carboxylic acid compound |
| C₃H₈O₃ (glycerol) | C₈ carboxylic acid compound |
| C₃H₈O₃ (glycerol) | C₉ carboxylic acid compound |
| C₃H₈O₃ (glycerol) | C₁₀ carboxylic acid compound |
| C₃H₈O₃ (glycerol) | C₁₁ carboxylic acid compound |
| C₃H₈O₃ (glycerol) | C₁₂ carboxylic acid compound |
| C₃H₈O₃ (glycerol) | C₁₄ carboxylic acid compound |
| C₃H₈O₃ (glycerol) | C₁₆ carboxylic acid compound |
| C₃H₈O₃ (glycerol) | C₁₈ carboxylic acid compound |
| C₃H₈O₃ (glycerol) | C₂₀ carboxylic acid compound |
| C₃H₈O₃ (glycerol) | C₂₂ carboxylic acid compound |
| C₃H₈O₃ (glycerol) | α-hydroxy acid |
| C₃H₈O₃ (glycerol) | α-amino acid |
| C₄H₁₀O₄ (erythritol) | C₂ carboxylic acid compound |
| C₄H₁₀O₄ (erythritol) | C₃ carboxylic acid compound |
| C₄H₁₀O₄ (erythritol) | C₅ carboxylic acid compound |
| C₄H₁₀O₄ (erythritol) | C₆ carboxylic acid compound |
| C₄H₁₀O₄ (erythritol) | C₇ carboxylic acid compound |
| C₄H₁₀O₄ (erythritol) | C₈ carboxylic acid compound |
| C₄H₁₀O₄ (erythritol) | C₉ carboxylic acid compound |
| C₄H₁₀O₄ (erythritol) | C₁₀ carboxylic acid compound |
| C₄H₁₀O₄ (erythritol) | C₁₁ carboxylic acid compound |
| C₄H₁₀O₄ (erythritol) | C₁₂ carboxylic acid compound |
| C₄H₁₀O₄ (erythritol) | C₁₄ carboxylic acid compound |
| C₄H₁₀O₄ (erythritol) | C₁₆ carboxylic acid compound |
| C₄H₁₀O₄ (erythritol) | C₁₈ carboxylic acid compound |
| C₄H₁₀O₄ (erythritol) | C₂₀ carboxylic acid compound |
| C₄H₁₀O₄ (erythritol) | C₂₂ carboxylic acid compound |
| C₄H₁₀O₄ (erythritol) | α-hydroxy acid |
| C₄H₁₀O₄ (erythritol) | α-amino acid |
| C₅H₁₂O₅ (xylitol) | C₂ carboxylic acid compound |
| C₅H₁₂O₅ (xylitol) | C₃ carboxylic acid compound |
| C₅H₁₂O₅ (xylitol) | C₅ carboxylic acid compound |
| C₅H₁₂O₅ (xylitol) | C₆ carboxylic acid compound |
| C₅H₁₂O₅ (xylitol) | C₇ carboxylic acid compound |
| C₅H₁₂O₅ (xylitol) | C₈ carboxylic acid compound |
| C₅H₁₂O₅ (xylitol) | C₉ carboxylic acid compound |
| C₅H₁₂O₅ (xylitol) | C₁₀ carboxylic acid compound |
| C₅H₁₂O₅ (xylitol) | C₁₁ carboxylic acid compound |
| C₅H₁₂O₅ (xylitol) | C₁₂ carboxylic acid compound |
| C₅H₁₂O₅ (xylitol) | C₁₄ carboxylic acid compound |
| C₅H₁₂O₅ (xylitol) | C₁₆ carboxylic acid compound |
| C₅H₁₂O₅ (xylitol) | C₁₈ carboxylic acid compound |
| C₅H₁₂O₅ (xylitol) | C₂₀ carboxylic acid compound |
| C₅H₁₂O₅ (xylitol) | C₂₂ carboxylic acid compound |
| C₅H₁₂O₅ (xylitol) | α-hydroxy acid |
| C₅H₁₂O₅ (xylitol) | α-amino acid |
| C₆H₁₄O₆ (sorbitol, mannitol) | C₂ carboxylic acid compound |
| C₆H₁₄O₆ (sorbitol, mannitol) | C₃ carboxylic acid compound |
| C₆H₁₄O₆ (sorbitol, mannitol) | C₅ carboxylic acid compound |
| C₆H₁₄O₆ (sorbitol, mannitol) | C₆ carboxylic acid compound |
| C₆H₁₄O₆ (sorbitol, mannitol) | C₇ carboxylic acid compound |
| C₆H₁₄O₆ (sorbitol, mannitol) | C₈ carboxylic acid compound |
| C₆H₁₄O₆ (sorbitol, mannitol) | C₉ carboxylic acid compound |
| C₆H₁₄O₆ (sorbitol, mannitol) | C₁₀ carboxylic acid compound |
| C₆H₁₄O₆ (sorbitol, mannitol) | C₁₁ carboxylic acid compound |
| C₆H₁₄O₆ (sorbitol, mannitol) | C₁₂ carboxylic acid compound |
| C₆H₁₄O₆ (sorbitol, mannitol) | C₁₄ carboxylic acid compound |
| C₆H₁₄O₆ (sorbitol, mannitol) | C₁₆ carboxylic acid compound |
| C₆H₁₄O₆ (sorbitol, mannitol) | C₁₈ carboxylic acid compound |
| C₆H₁₄O₆ (sorbitol, mannitol) | C₂₀ carboxylic acid compound |
| C₆H₁₄O₆ (sorbitol, mannitol) | C₂₂ carboxylic acid compound |
| C₆H₁₄O₆ (sorbitol, mannitol) | α-hydroxy acid |
| C₆H₁₄O₆ (sorbitol, mannitol) | α-amino acid |
| Inulin | C₂ carboxylic acid compound |
| Inulin | C₃ carboxylic acid compound |
| Inulin | C₅ carboxylic acid compound |
| Inulin | C₆ carboxylic acid compound |
| Inulin | C₇ carboxylic acid compound |
| Inulin | C₈ carboxylic acid compound |
| Inulin | C₉ carboxylic acid compound |
| Inulin | C₁₀ carboxylic acid compound |
| Inulin | C₁₁ carboxylic acid compound |
| Inulin | C₁₂ carboxylic acid compound |
| Inulin | C₁₄ carboxylic acid compound |
| Inulin | C₁₆ carboxylic acid compound |
| Inulin | C₁₈ carboxylic acid compound |
| Inulin | C₂₀ carboxylic acid compound |
| Inulin | C₂₂ carboxylic acid compound |
| Inulin | α-hydroxy acid |
| Inulin | α-amino acid |

In an alternative exemplary embodiment, the polyol ester is a compound comprising Formula (II):

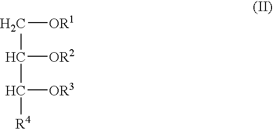

wherein:
R¹, R², and R³ are independently selected from the group consisting of hydrogen, an amino acid, and a carboxylic acid or a substituted carboxylic acid having from two to twenty-two carbon atoms;
R⁴ is hydrogen or (CH₂OR⁵)ₘ;
m is an integer from 1 to 3; and
R⁵ is independently selected from the group consisting of hydrogen, an amino acid, and a carboxylic acid or a substituted carboxylic acid having from two to twenty-two carbon atoms.

In a preferred embodiment for polyol esters having Formula (II), at least one of R¹, R² or R³ is a compound having Formula (I). An exemplary compound having Formula (I) is HMTBA. In one alternative of this embodiment, the other two of R¹, R² or R³ are carboxylic acids or substituted carboxylic acids not having Formula (I). In still another alternative of this embodiment, one of R¹, R² or R³ is a carboxylic acid or a substituted carboxylic acid not having Formula (I) and one of R¹, R² or R³ is an amino acid. In a further alternative of this embodiment, one of R¹, R² or R³ is a carboxylic acid or substituted carboxylic acid not having Formula (I) and the other of R¹, R² or R³ is hydrogen. In an additional alternative of this embodiment, two of R¹, R² or R³ are amino acids. In still an additional alternative of this embodiment, one of R¹, R² or R³ is an amino acid and one of R¹, R² or R³ is hydrogen. In another alternative of this embodiment, two of R¹, R² or R³ are hydrogen.

In another preferred embodiment for polyol esters having Formula (II), two of R¹, R² or R³ are compounds having Formula (I). An exemplary compound having Formula (I) is HMTBA. In one alternative of this embodiment, the other of R¹, R² or R³ is a carboxylic acid or a substituted carboxylic acid not having Formula (I). In an additional alternative of this embodiment, the other of R¹, R² or R³ is an amino acid. In still another alternative of this embodiment, the other of R¹, R² or R³ is hydrogen.

In yet another preferred embodiment for polyol esters having Formula (II), all three of R¹, R² or R³ are compounds having Formula (I). An exemplary compound having Formula (I) is HMTBA.

For each of the foregoing embodiments for polyol esters comprising Formula (II), R⁴ may be hydrogen. Alternatively, R⁴ may be (CH₂OR⁵)ₘ. In certain embodiments, m is one. In other embodiments, m is two. In additional embodiments, m is three. For embodiments where one or more of R¹, R² or R³ is a carboxylic acid or substituted carboxylic acid, suitable carboxylic acids or substituted carboxylic acids include any described in (I)(b). Typically, the carboxylic acid or substituted carboxylic acid may have from three to twenty-two carbon atoms or from three to twelve carbon atoms. Exemplary carboxylic acids include propionic acid, butanoic acid, octanoic acid, and decanoic acid. For embodiments where one or more of R¹, R² or R³ is an amino acid, exemplary amino acids include essential amino acids. Suitable polyol esters comprising Formula (II) are detailed in Table B.

TABLE B
Formula (II) Compounds

| R¹, R² or R³ | R¹, R² or R³ | R¹, R² or R³ | R⁴ |
|---|---|---|---|
| C₂-C₂₂ carboxylic | C₂-C₂₂ carboxylic | C₂-C₂₂ carboxylic | Hydrogen |
| acid compound | acid compound | acid compound | |
| C₂-C₂₂ carboxylic | C₂-C₂₂ carboxylic | Amino acid | Hydrogen |
| acid compound | acid compound | | |
| C₂-C₂₂ carboxylic | C₂-C₂₂ carboxylic | Formula (I) | Hydrogen |
| acid compound | acid compound | | |
| C₂-C₂₂ carboxylic | C₂-C₂₂ carboxylic | Hydrogen | Hydrogen |
| acid compound | acid compound | | |
| C₂-C₂₂ carboxylic | Amino acid | Amino acid | Hydrogen |
| acid compound | | | |
| C₂-C₂₂ carboxylic | Amino acid | Formula (I) | Hydrogen |
| acid compound | | | |
| C₂-C₂₂ carboxylic | Amino acid | Hydrogen | Hydrogen |
| acid compound | | | |
| C₂-C₂₂ carboxylic | Formula (I) | Formula (I) | Hydrogen |
| acid compound | | | |
| C₂-C₂₂ carboxylic | Formula (I) | Hydrogen | Hydrogen |
| acid compound | | | |
| C₂-C₂₂ carboxylic | Hydrogen | Hydrogen | Hydrogen |
| acid compound | | | |
| C₂-C₂₂ carboxylic | C₂-C₂₂ carboxylic | C₂-C₂₂ carboxylic | CH₂OR⁵ |
| acid compound | acid compound | acid compound | |
| C₂-C₂₂ carboxylic | C₂-C₂₂ carboxylic | Amino acid | CH₂OR⁵ |
| acid compound | acid compound | | |
| C₂-C₂₂ carboxylic | C₂-C₂₂ carboxylic | Formula (I) | CH₂OR⁵ |
| acid compound | acid compound | | |
| C₂-C₂₂ carboxylic | C₂-C₂₂ carboxylic | Hydrogen | CH₂OR⁵ |
| acid compound | acid compound | | |
| C₂-C₂₂ carboxylic | Amino acid | Amino acid | CH₂OR⁵ |
| acid compound | | | |
| C₂-C₂₂ carboxylic | Amino acid | Formula (I) | CH₂OR⁵ |
| acid compound | | | |
| C₂-C₂₂ carboxylic | Amino acid | Hydrogen | CH₂OR⁵ |
| acid compound | | | |
| C₂-C₂₂ carboxylic | Formula (I) | Formula (I) | CH₂OR⁵ |
| acid compound | | | |
| C₂-C₂₂ carboxylic | Formula (I) | Hydrogen | CH₂OR⁵ |
| acid compound | | | |
| C₂-C₂₂ carboxylic | Hydrogen | Hydrogen | CH₂OR⁵ |
| acid compound | | | |
| C₂-C₂₂ carboxylic | C₂-C₂₂ carboxylic | C₂-C₂₂ carboxylic | (CH₂OR⁵)₂ |
| acid compound | acid compound | acid compound | |
| C₂-C₂₂ carboxylic | C₂-C₂₂ carboxylic | Amino acid | (CH₂OR⁵)₂ |
| acid compound | acid compound | | |
| C₂-C₂₂ carboxylic | C₂-C₂₂ carboxylic | Formula (I) | (CH₂OR⁵)₂ |
| acid compound | acid compound | | |
| C₂-C₂₂ carboxylic | C₂-C₂₂ carboxylic | Hydrogen | (CH₂OR⁵)₂ |
| acid compound | acid compound | | |
| C₂-C₂₂ carboxylic | Amino acid | Amino acid | (CH₂OR⁵)₂ |
| acid compound | | | |
| C₂-C₂₂ carboxylic | Amino acid | Formula (I) | (CH₂OR⁵)₂ |
| acid compound | | | |
| C₂-C₂₂ carboxylic | Amino acid | Hydrogen | (CH₂OR⁵)₂ |
| acid compound | | | |
| C₂-C₂₂ carboxylic | Formula (I) | Formula (I) | (CH₂OR⁵)₂ |
| acid compound | | | |
| C₂-C₂₂ carboxylic | Formula (I) | Hydrogen | (CH₂OR⁵)₂ |
| acid compound | | | |
| C₂-C₂₂ carboxylic | Hydrogen | Hydrogen | (CH₂OR⁵)₂ |
| acid compound | | | |
| C₂-C₂₂ carboxylic | C₂-C₂₂ carboxylic | C₂-C₂₂ carboxylic | (CH₂OR⁵)₃ |
| acid compound | acid compound | acid compound | |
| C₂-C₂₂ carboxylic | C₂-C₂₂ carboxylic | Amino acid | (CH₂OR⁵)₃ |
| acid compound | acid compound | | |
| C₂-C₂₂ carboxylic | C₂-C₂₂ carboxylic | Formula (I) | (CH₂OR⁵)₃ |
| acid compound | acid compound | | |
| C₂-C₂₂ carboxylic | C₂-C₂₂ carboxylic | Hydrogen | (CH₂OR⁵)₃ |
| acid compound | acid compound | | |
| C₂-C₂₂ carboxylic | Amino acid | Amino acid | (CH₂OR⁵)₃ |
| acid compound | | | |
| C₂-C₂₂ carboxylic | Amino acid | Formula (I) | (CH₂OR⁵)₃ |
| acid compound | | | |
| C₂-C₂₂ carboxylic | Amino acid | Hydrogen | (CH₂OR⁵)₃ |
| acid compound | | | |
| C₂-C₂₂ carboxylic | Formula (I) | Formula (I) | (CH₂OR⁵)₃ |
| acid compound | | | |
| C₂-C₂₂ carboxylic | Formula (I) | Hydrogen | (CH₂OR⁵)₃ |
| acid compound | | | |
| C₂-C₂₂ carboxylic | Hydrogen | Hydrogen | (CH₂OR⁵)₃ |
| acid compound | | | |
| Amino acid | Amino acid | Amino acid | Hydrogen |
| Amino acid | Amino acid | Formula (I) | Hydrogen |
| Amino acid | Amino acid | Hydrogen | Hydrogen |
| Amino acid | Formula (I) | Formula (I) | Hydrogen |
| Amino acid | Formula (I) | Hydrogen | Hydrogen |
| Amino acid | Hydrogen | Hydrogen | Hydrogen |
| Amino acid | Amino acid | Amino acid | CH₂OR⁵ |
| Amino acid | Amino acid | Formula (I) | CH₂OR⁵ |
| Amino acid | Amino acid | Hydrogen | CH₂OR⁵ |
| Amino acid | Formula (I) | Formula (I) | CH₂OR⁵ |
| Amino acid | Formula (I) | Hydrogen | CH₂OR⁵ |
| Amino acid | Hydrogen | Hydrogen | CH₂OR⁵ |
| Amino acid | Amino acid | Amino acid | (CH₂OR⁵)₂ |
| Amino acid | Amino acid | Formula (I) | (CH₂OR⁵)₂ |
| Amino acid | Amino acid | Hydrogen | (CH₂OR⁵)₂ |
| Amino acid | Formula (I) | Formula (I) | (CH₂OR⁵)₂ |
| Amino acid | Formula (I) | Hydrogen | (CH₂OR⁵)₂ |
| Amino acid | Hydrogen | Hydrogen | (CH₂OR⁵)₂ |
| Amino acid | Amino acid | Amino acid | (CH₂OR⁵)₃ |
| Amino acid | Amino acid | Formula (I) | (CH₂OR⁵)₃ |
| Amino acid | Amino acid | Hydrogen | (CH₂OR⁵)₃ |
| Amino acid | Formula (I) | Formula (I) | (CH₂OR⁵)₃ |
| Amino acid | Formula (I) | Hydrogen | (CH₂OR⁵)₃ |
| Amino acid | Hydrogen | Hydrogen | (CH₂OR⁵)₃ |
| Formula (I) | Formula (I) | Formula (I) | Hydrogen |
| Formula (I) | Formula (I) | Hydrogen | Hydrogen |
| Formula (I) | Hydrogen | Hydrogen | Hydrogen |
| Formula (I) | Formula (I) | Formula (I) | CH₂OR⁵ |
| Formula (I) | Formula (I) | Hydrogen | CH₂OR⁵ |
| Formula (I) | Hydrogen | Hydrogen | CH₂OR⁵ |
| Formula (I) | Formula (I) | Formula (I) | (CH₂OR⁵)₂ |
| Formula (I) | Formula (I) | Hydrogen | (CH₂OR⁵)₂ |
| Formula (I) | Hydrogen | Hydrogen | (CH₂OR⁵)₂ |
| Formula (I) | Formula (I) | Formula (I) | (CH₂OR⁵)₃ |
| Formula (I) | Formula (I) | Hydrogen | (CH₂OR⁵)₃ |
| Formula (I) | Hydrogen | Hydrogen | (CH₂OR⁵)₃ |

In yet another alternative exemplary embodiment, the polyol ester is a glycerol ester comprising Formula (III):

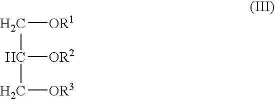

wherein:
R¹, R², and R³ are independently selected from the group consisting of hydrogen, a carboxylic acid or substituted carboxylic acid having from two to twenty-two carbon atoms, and an amino acid.

In a preferred embodiment for glycerol esters having Formula (III), at least one of R¹, R² or R³ is a compound having Formula (I). An exemplary compound having Formula (I) is HMTBA. In one alternative of this embodiment, the other two of R¹, R² or R³ are carboxylic acids or substituted carboxylic acids not having Formula (I). In still another alternative of this embodiment, one of R¹, R² or R³ is a carboxylic acid or a substituted carboxylic acid not having Formula (I) and one of R¹, R² or R³ is an amino acid. In a further alternative of this embodiment, one of R¹, R² or R³ is a carboxylic acid or substituted carboxylic acid not having Formula (I) and the other of R¹, R² or R³ is hydrogen. In an additional alternative of this embodiment, two of R¹, R² or R³ are amino acids. In still an additional alternative of this embodiment, one of R¹, R² or R³ is an amino acid and one of R¹, R² or R³ is hydrogen. In another alternative of this embodiment, two of R¹, R² or R³ are hydrogen.

In another preferred embodiment for glycerol esters having Formula (III), two of R¹, R² or R³ are compounds having Formula (I). An exemplary compound having Formula (I) is HMTBA. In one alternative of this embodiment, the other of R¹, R² or R³ is a carboxylic acid or a substituted carboxylic acid not having Formula (I). In an additional alternative of this embodiment, the other of R¹, R² or R³ is an amino acid. In still another alternative of this embodiment, the other of R¹, R² or R³ is hydrogen.

In yet another preferred embodiment for glycerol esters having Formula (III), all three of R¹, R² or R³ are compounds having Formula (I). An exemplary compound having Formula (I) is HMTBA.

For each of the foregoing embodiments for glycerol esters comprising Formula (III) where one or more of R¹, R² or R³ is a carboxylic acid or substituted carboxylic acid, suitable carboxylic acids or substituted carboxylic acids include any described in (I)(b). Typically, the carboxylic acid or substituted carboxylic acid may have from three to twenty-two carbon atoms or from three to twelve carbon atoms. Exemplary carboxylic acids include propionic acid, butanoic acid, octanoic acid, and decanoic acid. For embodiments where one or more of R¹, R² or R³ is an amino acid, exemplary amino acids include essential amino acids. Suitable glycerol esters comprising Formula (III) are detailed in Table C.

TABLE C
Formula (III) Compounds

| R¹, R² or R³ | R¹, R² or R³ | R¹, R² or R³ |
|---|---|---|
| C₂-C₂₂ carboxylic acid | C₂-C₂₂ carboxylic acid | C₂-C₂₂ carboxylic acid |
| compound | compound | compound |
| C₂-C₂₂ carboxylic acid | C₂-C₂₂ carboxylic acid | C₃-C₁₂ carboxylic acid |
| compound | compound | compound |
| C₂-C₂₂ carboxylic acid | C₂-C₂₂ carboxylic acid | Propionic acid |
| compound | compound | |
| C₂-C₂₂ carboxylic acid | C₂-C₂₂ carboxylic acid | Amino acid |
| compound | compound | |
| C₂-C₂₂ carboxylic acid | C₂-C₂₂ carboxylic acid | Formula (I) |
| compound | compound | |
| C₂-C₂₂ carboxylic acid | C₂-C₂₂ carboxylic acid | HMTBA |
| compound | compound | |
| C₂-C₂₂ carboxylic acid | C₂-C₂₂ carboxylic acid | Hydrogen |
| compound | compound | |
| C₂-C₂₂ carboxylic acid | C₃-C₁₂ carboxylic acid | C₃-C₁₂ carboxylic acid |
| compound | compound | compound |
| C₂-C₂₂ carboxylic acid | C₃-C₁₂ carboxylic acid | Propionic acid |
| compound | compound | |
| C₂-C₂₂ carboxylic acid | C₃-C₁₂ carboxylic acid | Amino acid |
| compound | compound | |
| C₂-C₂₂ carboxylic acid | C₃-C₁₂ carboxylic acid | Formula (I) |
| compound | compound | |
| C₂-C₂₂ carboxylic acid | C₃-C₁₂ carboxylic acid | HMTBA |
| compound | compound | |
| C₂-C₂₂ carboxylic acid | C₃-C₁₂ carboxylic acid | Hydrogen |
| compound | compound | |
| C₂-C₂₂ carboxylic acid | Propionic acid | Propionic acid |
| compound | | |
| C₂-C₂₂ carboxylic acid | Propionic acid | Amino acid |
| compound | | |
| C₂-C₂₂ carboxylic acid | Propionic acid | Formula (I) |
| compound | | |
| C₂-C₂₂ carboxylic acid | Propionic acid | Hydrogen |
| compound | | |
| C₂-C₂₂ carboxylic acid | Propionic acid | HMTBA |
| compound | | |
| C₂-C₂₂ carboxylic acid | Amino acid | Amino acid |
| compound | | |
| C₂-C₂₂ carboxylic acid | Amino acid | Formula (I) |
| compound | | |
| C₂-C₂₂ carboxylic acid | Amino acid | HMTBA |
| compound | | |
| C₂-C₂₂ carboxylic acid | Amino acid | Hydrogen |
| compound | | |
| C₂-C₂₂ carboxylic acid | Formula (I) | Formula (I) |
| compound | | |
| C₂-C₂₂ carboxylic acid | Formula (I) | HMTBA |
| compound | | |
| C₂-C₂₂ carboxylic acid | Formula (I) | Hydrogen |
| compound | | |
| C₂-C₂₂ carboxylic acid | HMTBA | HMTBA |
| compound | | |
| C₂-C₂₂ carboxylic acid | HMTBA | Hydrogen |
| compound | | |
| C₂-C₂₂ carboxylic acid | Hydrogen | Hydrogen |
| compound | | |
| C₃-C₁₂ carboxylic acid | C₃-C₁₂ carboxylic acid | C₃-C₁₂ carboxylic acid |
| compound | compound | compound |
| C₃-C₁₂ carboxylic acid | C₃-C₁₂ carboxylic acid | Propionic acid |
| compound | compound | |
| C₃-C₁₂ carboxylic acid | C₃-C₁₂ carboxylic acid | Amino acid |
| compound | compound | |
| C₃-C₁₂ carboxylic acid | C₃-C₁₂ carboxylic acid | Formula (I) |
| compound | compound | |
| C₃-C₁₂ carboxylic acid | C₃-C₁₂ carboxylic acid | Hydrogen |
| compound | compound | |
| C₃-C₁₂ carboxylic acid | C₃-C₁₂ carboxylic acid | HMTBA |
| compound | compound | |
| C₃-C₁₂ carboxylic acid | Butanoic acid | HMTBA |
| compound | | |
| C₃-C₁₂ carboxylic acid | Octanoic acid | HMTBA |
| compound | | |
| C₃-C₁₂ carboxylic acid | Decanoic acid | HMTBA |
| compound | | |
| Butanoic acid | Butanoic acid | HMTBA |
| Octanoic acid | Butanoic acid | HMTBA |
| Decanoic acid | Butanoic acid | HMTBA |
| Octanoic acid | Octanoic acid | HMTBA |
| Decanoic acid | Octanoic acid | HMTBA |
| Decanoic acid | Decanoic acid | HMTBA |
| C₃-C₁₂ carboxylic acid | Propionic acid | HMTBA |
| compound | | |
| C₃-C₁₂ carboxylic acid | Propionic acid | Amino acid |
| compound | | |
| C₃-C₁₂ carboxylic acid | Propionic acid | Formula (I) |
| compound | | |
| C₃-C₁₂ carboxylic acid | Propionic acid | Propionic acid |
| compound | | |
| C₃-C₁₂ carboxylic acid | Propionic acid | Hydrogen |
| compound | | |
| C₃-C₁₂ carboxylic acid | Amino acid | Amino acid |
| compound | | |
| C₃-C₁₂ carboxylic acid | Amino acid | Formula (I) |
| compound | | |
| C₃-C₁₂ carboxylic acid | Amino acid | HMTBA |
| compound | | |
| C₃-C₁₂ carboxylic acid | Amino acid | Hydrogen |
| compound | | |
| C₃-C₁₂ carboxylic acid | Formula (I) | Formula (I) |
| compound | | |
| C₃-C₁₂ carboxylic acid | Formula (I) | HMTBA |
| compound | | |
| C₃-C₁₂ carboxylic acid | Formula (I) | Hydrogen |
| compound | | |
| C₃-C₁₂ carboxylic acid | Hydrogen | Hydrogen |
| compound | | |
| C₃-C₁₂ carboxylic acid | HMTBA | HMTBA |
| compound | | |
| C₃-C₁₂ carboxylic acid | HMTBA | Hydrogen |
| compound | | |
| Butanoic acid | HMTBA | Hydrogen |
| Octanoic acid | HMTBA | Hydrogen |
| Decanoic acid | HMTBA | Hydrogen |
| Butanoic acid | HMTBA | HMTBA |
| Octanoic acid | HMTBA | HMTBA |
| Decanoic acid | HMTBA | HMTBA |
| Propionic acid | Propionic acid | Propionic acid |
| Propionic acid | Propionic acid | Amino acid |
| Propionic acid | Propionic acid | Formula (I) |
| Propionic acid | Propionic acid | HMTBA |
| Propionic acid | Propionic acid | Hydrogen |
| Propionic acid | Amino acid | Amino acid |
| Propionic acid | Amino acid | Formula (I) |
| Propionic acid | Amino acid | HMTBA |
| Propionic acid | Amino acid | Hydrogen |
| Propionic acid | Formula (I) | Formula (I) |
| Propionic acid | Formula (I) | HMTBA |
| Propionic acid | Formula (I) | Hydrogen |
| Propionic acid | HMTBA | HMTBA |
| Propionic acid | HMTBA | Hydrogen |
| Propionic acid | Hydrogen | Hydrogen |
| Amino acid | Amino acid | Amino acid |
| Amino acid | Amino acid | Formula (I) |
| Amino acid | Amino acid | HMTBA |
| Amino acid | Amino acid | Hydrogen |
| Amino acid | Formula (I) | Formula (I) |
| Amino acid | Formula (I) | HMTBA |
| Amino acid | Formula (I) | Hydrogen |
| Amino acid | HMTBA | HMTBA |
| Amino acid | HMTBA | Hydrogen |
| Amino acid | Hydrogen | Hydrogen |
| Formula (I) | Formula (I) | Formula (I) |
| Formula (I) | Formula (I) | HMTBA |
| Formula (I) | Formula (I) | Hydrogen |
| Formula (I) | HMTBA | HMTBA |
| Formula (I) | HMTBA | Hydrogen |
| Formula (I) | Hydrogen | Hydrogen |
| HMTBA | HMTBA | HMTBA |
| HMTBA | HMTBA | Hydrogen |
| HMTBA | Hydrogen | Hydrogen |

(II) Process for Making Polyol Ester Compounds

The invention further provides a process for the preparation of polyol ester compounds. Generally speaking, the process involves combining the reactants, i.e., a polyol and a carboxylic acid, substituted carboxylic acid or amino acid, in the presence of a catalyst, and heating the reaction mixture to a sufficient temperature to form a polyol ester. Suitable polyols, carboxylic acids, substituted carboxylic acids, and amino acids are detailed in (I) above.

The reactants, i.e., the polyol and the carboxylic acid compound, are generally combined to form a reaction mixture. The molar ratio of polyol to carboxylic acid compound may range from about 0.01:1 to about 50:1. In one embodiment, the ratio of polyol to carboxylic acid compound may be about 0.05:1. In another embodiment, the ratio of polyol to carboxylic acid compound may be about 10:1.

The polyol of the reaction mixture may comprise a polyol having the D-configuration or the L-configuration, or a racemic or other mixture of the D- and L-isomers. The carboxylic acid of the reaction mixture may comprise a carboxylic acid having the D-configuration or the L-configuration, or a racemic or other mixture of the D- and L-isomers. The substituted carboxylic acid, such as a α-hydroxy carboxylic acid or an α-amino acid, of the reaction mixture may comprise a substituted carboxylic acid having the D-configuration or the L-configuration, or a racemic or other mixture of the D- and L-isomers.

The reactants are generally combined in the presence of an organic solvent. The solvent can and will vary depending upon the nature of the reactants. Suitable solvents include, but are not limited to, toluene, benzene, hexane, cyclohexane, ethyl acetate, tetrahydrofuran, acetonitrile, dimethylformamide, dioxane, and dimethylsulfoxide.

A catalyst is generally added to a reaction mixture to increase the rate of the reaction. In a preferred embodiment, the catalyst is a triflate-substituted catalyst. Triflate, or trifluoromethanesulfonate, is an extremely stable polyatomic ion. Generally speaking, triflate-substituted catalysts are potent and efficient catalysts that are noncorrosive and easy to handle. The catalyst may be scandium triflate, stannous triflate, lanthamide triflate, or yttrium triflate. The molar ratio of polyol and carboxylic acid compound to triflate-substituted catalyst may range from about 300:1 to about 5000:1. In one embodiment, the molar ratio of polyol and carboxylic acid compound to triflate-substituted catalyst may be about 1000:1. In another embodiment, the molar ratio of polyol and carboxylic acid compound to triflate-substituted catalyst may be about 3500:1.

The reaction mixture is generally mixed or stirred continuously and heated to an appropriate temperature to promote formation of an ester bond between a hydroxyl group of the polyol and a carboxyl group of the carboxylic acid compound. The reaction mixture may be heated to a temperature ranging from about 75° C. to about 140° C. In one embodiment, the reaction mixture may be heated to about 130° C. In another embodiment, the reaction mixture may be heated to about 110° C. In still another embodiment, the reaction mixture may be heated to about 85° C.

Specific hydroxyl groups of the polyol may be protected to direct the reaction to a preselected hydroxyl group of the polyol. The use of protecting groups is well known in the art for protecting hydroxyl groups against reaction during a synthetic procedure and many such protecting groups are known, e.g., T. H. Greene and P. G. M. Wuts, Protective Groups in Organic Synthesis, 3rd edition, John Wiley & Sons, New York (1999), and references therein. Typical hydroxyl protecting groups include, but are not limited to, methoxymethyl, methylthiomethyl, trialkylsilyl and diarylalkylsilyl groups (e.g. tert-butyldimethylsilyl, tert-butyldiphenylsilyl or trimethylsilyl) and tetrahydropyranyl. Of the above-listed groups, tert-butyldimethylsilyl and tert-butyldiphenylsilyl are generally selective for the hydroxyl groups of primary alcohols. Specific protecting reagents may be used to protect two hydroxyl groups (i.e., diols) of a compound by essentially linking these groups. Isopropylidenes, such as acetonide, generally protect 1,2-diols over 1,3-diols. Benzylidene acetals, such as benzaldehye, generally protect 1,3-diols over 1,2-diols. Di-tert-butylsilylene and 1,3-(1,1,3,3)-tetraisopropyldisiloxanylidene generally protects 1,3- and 1,4-diols over 1,2-diols. The addition and removal of hydroxyl protecting groups may be achieved by conventional methods, for example, as described in Greene and Wuts (1999). Likewise, sensitive groups on some substituted carboxylic acids or α-amino acids may need to be protected (and deprotected) using conventional methods. The sequence of reactions for protection and esterification can and will vary depending upon the target compound to be synthesized.

If the polyol is glycerol, then the reactivity difference between the more reactive C1 and C3 primary hydroxyls versus the less reactive C2 secondary hydroxyl may be exploited to synthesize the desired glycerol ester compound. For example, the two primary alcohols of glycerol may be linked and protected with benzaldehyde to form the acetal, cis-1,3-O-benzylidene glycerol, such that the remaining secondary hydroxyl group may be esterified. Alternatively, the commercially available acetone ketal of glycerol, solketal (2,2-dimethyl-1,3-dioxolane-4-methanol) may be used as a reactant. In solketal, two adjacent hydroxyl groups of glycerol are linked and protected, and the remaining terminal hydroxyl group may be reacted with a carboxylic acid compound. After deprotection, the resultant monoester product may be further reacted and/or protected to form a glycerol diester or a glycerol triester.

The polyol ester reaction product may be isolated and purified from the reaction mixture using methods known to a skilled chemist. For example, the solvent may be removed by rotary evaporation, and the excess reactants may be removed by vacuum distillation. The reaction product may be analyzed by chromatography (GC) or gas chromatography-mass spectrometry (GC-MS). Conventional electron impact mass spectrometry (EI-MS) may be used to verify product identity.

(III) Food, Food Ingredients, and Feed Compositions

The invention provides food, food ingredients, and feed compositions comprising a polyol ester compound or a mixture of polyol ester compounds. The polyol ester compounds are generally designed to deliver easily absorbable nutrients to the intestine. The ester linkages are generally resistant to degradation in the acidic stomach of a monogastric animal or degradation by ruminal microorganisms in a ruminant. Once the polyol ester compounds enter the small intestine, however, intestinal enzymes, such as lipases and esterases, may hydrolyze the ester bonds. The intestinal cells may readily absorb the released carboxylic acid compounds. Simple polyols, such as glycerol are readily absorbed, whereas more complex polyols may have to be digested by enzymes in the small intestine or microbes in the large intestine before the breakdown products may be absorbed.

The food or feed compositions may also provide antimicrobial activity. Without being bound to any particular theory, hydrolysis of the ester linkages of the polyol esters and the concomitant release of carboxylic acid compounds generally leads to acidification of the luminal contents of the intestine. Microorganisms, such certain species or stains of Escherichia coli, Clostridia, and Campylobacter, may be sensitive to decreased pH levels or increased levels of free carboxylic acid compounds. As used herein, the term “inhibit” when used in phrases such as “inhibiting bacteria” means any one or more of (a) killing bacteria or mold; (b) any decrease in growth of the bacteria or mold, which may be measured in terms of colony counts; (c) any decrease in the concentration of bacteria or mold; or (d) the inability of bacteria or mold to grow on a particular selection medium. Each of these may be determined, for instance, by comparing the bacterial or fungal colony counts or concentration of bacteria or mold present in the absence of the application of the methods of the present invention with the bacterial or fungal colony counts or concentration of bacteria or mold after application of the methods of the present invention. Generally speaking, application of suitable bactericides or fungicides will show a ten-fold difference in colony counts.

Animals for which the food, food ingredients and/or feed compositions described herein may be provided include humans, ruminants such as dairy cows, lactating dairy cows, dairy calves, beef cattle, sheep, and goats; aquaculture such as fish and crustaceans (including, but not limited to, salmon, shrimp, carp, tilapia and shell fish); livestock such as swine and horses; poultry such as chickens, turkeys, and hatchlings thereof; and companion animals such as dogs and cats.

As will be appreciated by a skilled artisan, the concentration of polyol esters of the invention in a particular food, food ingredient and/or feed composition can and will vary without departing from the scope of the invention. Generally, the concentration of polyol ester in the compositions described herein is between about 0.01% and about 5% by weight. In various preferred embodiments, the concentration is between 0.01% and about 4% by weight; between 0.02% and about 3% by weight; between 0.03% and about 2% by weight; between 0.04% and about 1% by weight; between about 0.05% and about 0.6% by weight; and between about 0.06% and about 0.5% by weight.

The exact formulation of the above-mentioned animal feed composition is not critical to the present invention. Feed ingredients are selected according to the nutrient requirements of the particular animal for which the feed is intended; these requirements depend, interalia, upon the age and stage of development of the animal, the sex of the animal, and other factors. Feed ingredients may be grouped into eight classes on the basis of their composition and their use in formulating diets: dry forages and roughages; pasture, range plants and forages fed fresh; silages; energy feeds; protein supplements; mineral supplements; vitamin supplements; and additives. See National Research Council (U.S.) Subcommittee on Feed Composition, United States-Canadian Tables of Feed Composition, 3d rev., National Academy Press, pp. 2,145 (1982). These classes are, to a certain extent, arbitrary, as some feed ingredients could be classified in more than one class. Typically, a feed formulation will also depend upon the costs associated with each ingredient, with the least-expensive composition of ingredients that gives the needed nutrients being the preferred formulation.

Silage is a forage product that is produced from the harvest, storage and fermentation of green forage crops such as corn and grain sorghum plants. These crops are chopped, stems and all, before the grain is ready for harvest. The plant material is stored in silos, storage bags, bunkers or covered piles causing the material to ferment, thereby lowering the pH and preserving the plant material until it can be fed. The ensiled forage is subject to changes in pH, temperature, and oxygen levels. As noted above, feed formulations depend in part upon the age and stage of development of the animal to be fed. Leeson and Summers (Nutrition of the Chicken, 4^{th} ed., pp. 502-510, University Books 2001)) describe several representative poultry diets for pullets, layers, broilers and broiler breeders. For example, most chicken diets contain energy concentrates such as corn, oats, wheat, barley, or sorghum; protein sources such as soybean meal, other oilseed meals (e.g., peanut, sesame, safflower, sunflower, etc.), cottonseed meal, animal protein sources (meat and bone meal, dried whey, fish meal, etc.), grain legumes (e.g., dry beans, field peas, etc.), and alfalfa; and vitamin and mineral supplements, if necessary (for instance, meat and bone meal is high in calcium and phosphorous, and thus these minerals do not need to be supplemented in a feed ration containing meat and bone meal). The relative amounts of the different ingredients in poultry feed depend in part upon the production stage of the bird. Starter rations are higher in protein, while grower and finisher feeds can be lower in protein since older birds require less protein. Model diets for swine and other animals are also available, and may be modified according to the particular needs of the animal(s) to be fed.

The polyol ester compounds may be formulated as liquids, emulsions, or dry or powdered supplements to be added to other foods, such as grains, protein products, and mixtures thereof. The dry feed supplement may be uniformly dispersed throughout a dry or liquid food. Feed compositions may also be provided as aqueous formulations. An aqueous formulation may be a solution or an emulsion. The aqueous formulation may be added directly to the drinking water of an animal or it may be mixed into or applied to a dry or liquid food. The polyol ester compounds may be mixed with the other ingredients in the feed, such as the corn, soybean meal, other feed supplements, etc., as the feed is being formulated. Alternatively, the polyol ester compounds may be applied to a pre-mixed or pre-pelleted feed.

### Definitions

The term “acyl” denotes a radical having the general formula RCO—, provided after the removal of a hydroxyl group from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl. Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “carboxylic acid” used herein refers to organic acids comprising hydrocarbon groups that contain a carboxyl group (COOH). The hydrocarbon moiety consists exclusively of the elements carbon and hydrogen. Carboxylic acids may have straight chains (aliphatic) of hydrocarbyl groups, or they may be aromatic carboxylic acids, as well as some alicyclic carboxylic acids (i.e., both aliphatic and cyclic). Straight chain aliphatic carboxylic acids preferably have 3 to 24 carbons (including the terminal carboxyl carbon). The hydrocarbon chain of an aliphatic carboxylic acid may be saturated (i.e., the carbon atoms have all the hydrogen atoms they can hold) and contain no double bonds between the carbons. Alternatively, the hydrocarbon chain may be unsaturated and contain one or more double bonds between the some of the carbons. Unsaturated carboxylic acids may assume cis or trans configurations, which refer to the orientation of the hydrogen atoms with respect to the double bond. Cis means “on the same side” and trans means “across” or “on the other side”.

An “essential amino acid” is an amino acid that cannot be synthesized by an organism and must be supplied as part of its diet. It is generally recognized that ten amino acids are essential for humans and animals. The essential amino acids are arginine, histidine, isoleucine, leucine, lysine, methionine, phenylalanine, threonine, tryptophan, and valine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

“HMTBA” stands for 2-hydroxy-4-(methylthio)butanoic acid (sold under the trade name ALIMET® by Novus International, Inc., St. Louis, Mo.).

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “insulin” refers to a type of plant oligosaccharide mainly comprising fructose units, linked by β-(2,1) glycosidic bonds, and typically having a terminal glucose unit. The simplest insulin has two fructose units and one glucose unit.

The term “organic acid derivative” refers to a derivative of any suitable organic acid resulting from removal of the carboxyl function from the acid. Preferably, the organic acid has from one to eight carbon atoms. Suitable organic acid derivatives include, but are not limited to, derivatives of formic acid, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, and glutaric acid.

The term “polyol” is used in its broadest sense to encompass a compound having at least one accessible hydroxyl group. Generally, the compound may have three or more hydroxyl groups. As used herein, those having three hydroxyl groups are glycerols; those having four to six hydroxyl groups are called sugar alcohols (e.g., erythritol, xylitol, sorbitol, mannitol); and those with many more hydroxyl groups include oligosaccharides and polysaccharides (e.g., insulin).

The term “substituted carboxylic acid” used herein refers to substitutions within the hydrocarbyl chain of a straight chain aliphatic carboxylic acid. Hydrocarbyl moieties may be substituted with at least one atom, including the substitution of a carbon atom with a heteroatom such as nitrogen, oxygen, silicon, phosphorous, boron, sulfur, or a halogen atom. Substitutions may also include hydrocarbyl moieties, such as alkyl, alkenyl, alkynyl, and aryl moieties, with these moieties having one to 20 carbon atoms. Other substituted moieties include hydrocarbyloxy, such as acyloxy, alkoxy, alkenoxy, alkynoxy, aryloxy, hydroxy, protected hydroxy, keto, acyl, acyloxy, nitro, amino, amido, cyano, thiol, ketals, acetals, heterocyclo, esters and ethers. Dicarboxylic acids contain an additional carboxyl group at the other end of the molecule. α-Hydroxy acids are another type of substituted carboxylic acid; α-hydroxy acids generally have a hydroxyl group on the alpha carbon atom (i.e., the carbon adjacent to the terminal carbonyl carbon). α-Amino acids, which have an amino group on the alpha carbon, are also substituted carboxylic acids.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate various embodiments of the invention.

### Example 1

### Synthesis of Glycerol Esters of HMTBA

Glycerol was esterified with the α-hydroxy acid, 2-hydroxy-4-(methylthio)butanoic acid (HMTBA) using a one-step process. For this, 22.4 g (0.13 mole at 89% assay) of HMTBA was mixed with 100 ml of methylene chloride in a 250-ml Erlenmeyer flask and stirred for 10 minutes. The mixture was filtered by suction through a fine glass frit and the methylene chloride was removed by rotary evaporation. The residue was transferred to a four-neck, round-bottom flask with a stir bar. Then, 0.66 g (0.00134 mole) of scandium trifluoromethane sulfonate [Sc(SO₃CF₃)₃], 98 ml (10-fold molar excess) of glycerol, and 150 ml of toluene were added. The flask was attached to a thermocouple, a solvent trap with a reflex condenser, and a heating mantle. The mixture was stirred and heated to 112° C. The reaction was followed by measuring the volume of reaction water and the volume of residual HMTBA water that were azeotropically removed from the reaction. In a typical reaction of this scale, approximately 2.3 g of reaction water and 2.5 g of residual HMTBA water were removed. Upon completion of the reaction, the reaction mixture was cooled, transferred to a single neck flask, and the toluene was removed by rotary evaporation. The residue was dissolved in 150 ml of acetone, the solution was filtered, and the acetone was removed from the filtrate by rotary evaporation. The residue was transferred to a vacuum distillation system and excess glycerol was removed by vacuum distillation.

The product was analyzed by gas chromatography (GC) and gas chromatography-mass spectrometry (GC-MS). For this, 5 μl of the product sample was mixed with 200 μl of tetrahydrofuran (THF) and 1 ml of bis(trimethylsilyl)trifluoroacetamide (BSTFA). If the sample still contained significant amounts of glycerol, then methanol was substituted for THF. The mixture was shaken and allowed to stand for two hours before injection. A 30 m×0.25 mm×0.25 micron Rtx-1 (dimethyl polysiloxane) GC column was used. Conventional electron impact mass spectrometry (EI-MS) was used to verify product identity.

The GC-MS analysis revealed that both the monoester and the diester of HMTBA were formed during this reaction. The ratio of monoester to diester was 27:9 at the completion of the reaction, but prior to isolation of the reaction products. Equal amounts of the 1-monoester and the 2-monoester were formed. The ratio of the diester isomers was about 5:2, but it is not known whether the more abundant species is the 1,2-diester or the 1,3-diester.

### Example 2

### Synthesis of Glycerol Esters of Propionate

Glycerol was esterified with propionic acid by mixing 31.5 ml (0.27 mole) of glycerol and 0.6 g (0.0012 mole) of Sc(OCF₃SO₂—)₃ in a 500-ml three-neck, round-bottom flask with a stir bar. Then 300 ml of propionic acid and 50 ml of toluene were added. The flask was attached to a thermocouple, a solvent trap with a reflex condenser, and a heating mantle. The mixture was stirred and heated to 129° C. The reaction was followed by measuring the volume of water azeotropically removed from the reaction. In a typical reaction of this scale, approximately 14.7 g of reaction water was removed. The reaction was also followed by GC. For this, the reaction was cooled, and 50 μl of the reaction solution was combined with 1 ml of BSTFA. The mixture was allowed to stand at room temperature for 2 hours before injection into the GC.

Upon completion of the reaction, the reaction mixture was cooled, transferred to a single neck flask, and the toluene was removed by rotary evaporation. The residue was transferred to a vacuum distillation system and excess propionic acid was removed by vacuum distillation. The crude product was transferred to a suitable flask and distilled under high vacuum. Analysis of the reaction products by GC and GC-MS revealed that the most abundant species was the triester of propionoate, with a yield of 94.3% at 97.7% purity. A small amount of the diester was present (0.7%), and only a trace of the monoester was present.

### Example 3

### Synthesis of Glycerol Diesters of HMTBA

To increase the yield of diesters of HMTBA, the synthesis protocol was modified slightly. First, HMTBA was first treated with dioxane to remove bisulfate. For this, 56.0 g of HMTBA (0.332 mole at 89% assay) was mixed with 200 ml of 1,4-dioxane and filtered through a medium glass frit and the filtrate was retained (and the solvent was not removed). Next, 1.63 g (0.00332 mole) of Sc(OCF₃SO₂—)₃, 250 ml of glycerol (10-fold molar excess), and 400 ml of cyclohexane were added to a 1-liter four-neck, round-bottom flask containing a stir bar. The cyclohexane served as a solvent as well as an azeotrope for the water formed during the reaction. A thermocouple, a solvent trap with a reflex condenser and an argon inlet, and a 250-ml addition funnel were attached to the flask. The HMTBA filtrate was placed in the addition funnel. The reaction mixture was heated to reflux (about 85° C.) and aliquots of the HMTBA filtrate were added via the addition funnel at intervals over the course of the reaction. The rate of addition of the HMTBA filtrate was directly proportional to the amount of water formed. The reaction process was monitored by GC; and the reaction was continued until water formation was no longer observed. The reaction mix was cooled and purified by vacuum distillation to remove the dioxane, cyclohexane, and excess glycerol. The resultant reaction residue was analyzed by GC-MS as described in Example 1. The reaction residue contained 15.6% glycerol, 58.5% monoester, and 18.5% diester.

To convert more of the monoesters to diesters, the reaction residue was reacted again with additional HMTBA. The reaction residue (57.6 g) was added to a reaction flask, along with 350 ml of cyclohexane. Approximately 58 g of HMTBA was treated with 200 ml of 1,4-dioxane as described above, and placed in an addition funnel attached to the reaction flask. The amount of HMTBA used was calculated to be the molar concentration needed to react with the excess glycerol and monoester to theoretically yield 100% diester. The reaction mixture was stirred and heated to reflux (about 85° C.) and the HMTBA was added gradually as the reaction water formed. The reaction was monitored as described above. The final reaction mix contained a precipitate, which was presumably 7 the catalyst. The upper layers of the final reaction mix contained about 60% monoester and about 26% diester. Although these reaction conditions may still need to be modified, they gave a much higher yield of glycerol diesters of HMTBA than the conditions used in Example 1.

The reaction conditions described above should yield glycerol 1,3-diesters of HMTBA because the primary C1 and C3 hydroxyl groups are generally more reactive than the secondary C2 hydroxyl group. To synthesize 1,2-diesters of HMTBA, one of the primary hydroxyl groups of glycerol may be protected with either tert-butyldimethylsilyl or tert-butyldiphenylsilyl such that the two remaining hydroxyl groups may be reacted with HMTBA in the presence of a catalyst (e.g., Sc(OCF₃SO₂⁻)₃) under appropriate reactions conditions. Upon removal of the protectant, GC and/or GC-MS analyses may be used to confirm that the major product is the 1,2-diester.

### Example 4

### Triesterification of Glycerol with HMTBA

Triglycerides comprising three acyl radicals from HMTBA, may be synthesized by combining a 10- to 15-fold molar excess of HMTBA with glycerol, Sc(OCF₃SO₂—)₃, and solvent and reacting under appropriate conditions. GC and/or GC-MS analyses may be used to monitor the reaction and confirm the identity of the reaction products.

### Example 5

### Diesterification of Glycerol with HMTBA and Either Butanoic, Octanoic, or Decanoic Acid

Diesters of glycerol comprising one acyl radical from HMTBA and one acyl radical from either butanoic acid, octanoic acid, or decanoic acid may be synthesized using the methods of the invention. Equal amounts of HMTBA and octanoic acid (or HMTBA and octanoic acid or HMTBA and decanoic acid) may be combined with optimal amounts of glycerol, Sc(OCF₃SO₂⁻)₃, and solvent (e.g., toluene). The mixture may be carried out and monitored as described in Example 3. Since the C1 and C3 primary hydroxy groups are more reactive then the C2 hydroxyl group, the formation of diesters (i.e., 1,3-diesters) should be favored. Alternatively, commercially available monoesters of glycerol (glycerol C4 monoester, glycerol C8 monoester, or glycerol C10 monoester) may be used as starting material rather than glycerol and the appropriate free acid.

### Example 6

### Triesterification of Glycerol with Two Molecules of HMTBA and Either Butanoic, Octanoic, or Decanoic Acid

To synthesize triesters of glycerol comprising acyl radicals from two molecules of HMTBA and one acyl radical form either butanoic acid, octanoic acid, or decanoic acid, it may advantageous to protect the primary hydroxyl groups such that the less reactive secondary hydroxyl group (C2) may form an ester bond first. Accordingly, benzaldehyde may be used to link the C1 and C3 hydroxyl groups. The remaining C2 hydroxyl group may be reacted under appropriate conditions with butanoic acid (or octanoic acid or decanoic acid) in the presence of the catalyst Sc(OCF₃SO₂⁻)₃ to form an ester linkage. After deprotection, the primary hydroxyl groups may be reacted with a molar excess of HMTBA (and catalyst) to form the final two ester linkages. Alternatively, commercially available monoesters of glycerol (glycerol C4 monoester, glycerol C8 monoester, or glycerol C10 monoester) may be used as the starting material rather than glycerol and the appropriate free acid. A molar excess of HMTBA may be reacted with the glycerol monoester under the appropriate conditions to form the triester.

### Example 7

### Triesterification of Glycerol with HMTBA, Octanoic Acid, and Decanoic Acid

Triglycerides comprising an acyl radical from HMTBA, an acyl radical from octanoic acid, and an acyl radical from decanoic acid may be synthesized by reacting equal amounts of HMTBA, octanoic acid, and decanoic with optimal amounts of glycerol, Sc(OCF₃SO₂⁻)₃, and solvent under appropriate reaction conditions. Alternatively, it may be advantageous to react the less reactive secondary hydroxyl group first. For this, the primary hydroxyl groups may be protected with benzaldehyde, such that the secondary hydroxyl group may be reacted with HMTBA (which may require a different solvent than the C8 and C10 acids). After deprotection, the primary hydroxyl groups may be reacted with equal amounts of octanoic acid and decanoic acid. In another iteration, commercially available glycerol diesters comprising C8 and C10 groups may be used as the starting material.

### Example 8

### Triesterification of Glycerol with HMTBA, Butanoic Acid, and Decanoic Acid

Triglycerides comprising an acyl radical from HMTBA, an acyl radical from butanoic acid, and an acyl radical from decanoic acid may be synthesized by reacting equal amounts of HMTBA, butanoic acid, and decanoic with optimal amounts of glycerol, Sc(OCF₃SO₂⁻)₃, and solvent under appropriate reaction conditions. Alternatively, it may be advantageous to react the less reactive secondary hydroxyl group first. For this, the primary hydroxyl groups may be protected with benzaldehyde, such that the secondary hydroxyl group may be reacted with decanoic acid (which may require a different solvent than HMTBA and butanoic acid). After deprotection, the primary hydroxyl groups may be reacted with equal amounts of HMTBA and butanoic acid. In another iteration, commercially available glycerol diesters comprising C4 and C10 groups may be used as the starting material.

### Example 9

### Triesterification of Glycerol with HMTBA and Two Molecules of Octanoic Acid

Triglycerides comprising one acyl radical from HMTBA and two acyl radical from octanoic acid may be synthesized by reacting a 2:1 ratio of octanoic acid and HMTBA with optimal amounts of glycerol, Sc(OCF₃SO₂⁻)₃, and solvent under appropriate reaction conditions. Alternatively, it may be advantageous to react the less reactive secondary hydroxyl group first. For this, the primary hydroxyl groups may be protected with benzaldehyde, such that the secondary hydroxyl group may be reacted with HMTBA. After deprotection, the primary hydroxyl groups may be reacted with octanoic acid. In another iteration, commercially available glycerol C10 diesters may be used as the starting material.

What is claimed is:

1. A diester or triester of a compound comprising Formula (III): 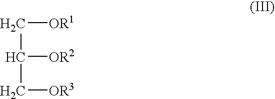
wherein:
R¹, R², and R³ are independently selected from the group consisting of hydrogen, a carboxylic acid or substituted carboxylic acid having from two to twenty-two carbon atoms, and an amino acid; provided that only one of R¹, R², and R³ may be hydrogen; and
provided that at least one of R¹, R² or R³ is a compound having Formula (I): 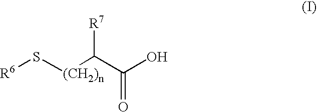
wherein:
n is an integer from 0 to 2;
R⁶ is alkyl group having from one to four carbon atoms;
R⁷ is selected from the group consisting of hydroxyl, amino, —OCOR⁸, or —NHCOR⁸; and
R⁸ is an organic acid derivative.

2. The compound of claim 1, wherein R⁶ is methyl or ethyl and R⁷ is hydroxyl or amino.

3. The compound of claim 1, wherein the compound having Formula (I) is 2-hydroxy-4-(methylthio)butanoic acid.

4. The compound of claim 1, wherein one of R¹, R², and R³ is 2-hydroxy-4-(methylthio)butanoic acid.

5. The compound of claim 4, wherein at least one of R¹, R², and R³ are carboxylic acids or substituted carboxylic acids having from three to twelve carbon atoms.

6. The compound of claim 4, wherein at least one of R¹, R², and R³ are selected from the group consisting of propionic acid, butanoic acid, octanoic acid, and decanoic acid.

7. The compound of claim 4, wherein one of R¹, R², or R³ is a carboxylic acid or substituted carboxylic acid having from three to twelve carbon atoms and one of R¹, R², or R³ is an amino acid.

8. The compound of claim 4, wherein one of R¹, R², or R³ is selected from the group consisting of propionic acid, butanoic acid, octanoic acid, and decanoic acid.

9. The compound of claim 4, wherein two of R¹, R², or R³ are amino acids.

10. The compound of claim 4, wherein two of R¹, R², or R³ are selected from the group consisting of arginine, histidine, isoleucine, leucine, lysine, methionine, phenylalanine, threonine, tryptophan, and valine.

11. The compound of claim 4, wherein one of R¹, R², or R³ is an amino acid and one of R¹, R², or R³ is hydrogen.

12. The compound of claim 4, wherein one of R¹, R², or R³ is selected from the group consisting of arginine, histidine, isoleucine, leucine, lysine, methionine, phenylalanine, threonine, tryptophan, and valine.

13. The compound of claim 1, wherein two of R¹, R², and R³ are 2-hydroxy-4-(methylthio)butanoic acid.

14. The compound of claim 13, wherein one of R¹, R², or R³ is a carboxylic acid or substituted carboxylic acid having from three to twelve carbon atoms.

15. The compound of claim 13, wherein one of R¹, R², and R³ is selected from the group consisting of propionic acid, butanoic acid, octanoic acid, and decanoic acid.

16. The compound of claim 13, wherein one of R¹, R², or R³ is an amino acid.

17. The compound of claim 16, wherein the amino acid is selected from the group consisting of arginine, histidine, isoleucine, leucine, lysine, methionine, phenylalanine, threonine, tryptophan, and valine.

18. The compound of claim 1, wherein each of R¹, R², and R³ is 2-hydroxy-4-(methylthio)butanoic acid.

19. A process for the preparation of a polyol ester, the process comprising:
a. combining a polyol and at least one carboxylic acid compound in the presence of a triflate-substituted catalyst to form a reaction mixture; and
b. heating the reaction mixture to an appropriate temperature to form the polyol ester.

20. The process of claim 19, wherein the triflate-substituted catalyst is selected from the group consisting of scandium triflate, stannous triflate, lanthamide triflate, and yttrium triflate.

21. The process of claim 20, wherein the polyol is selected from the group consisting glycerol, sorbitol, xylitol, mannitol, and insulin, and the carboxylic acid compound is selected from the group consisting of a carboxylic acid or substituted carboxylic acid having from three to twenty-two carbon atoms and an amino acid.

22. The process of claim 19, wherein the polyol is glycerol and the carboxylic acid compound is selected from the group consisting of 2-hydroxy-4-(methylthio)butanoic acid, a carboxylic acid or substituted carboxylic acid having from three to twelve carbon atoms, and an amino acid.

23. The process of claim 19, wherein the polyol is glycerol and the carboxylic acid compound is 2-hydroxy-4-(methylthio)butanoic acid.

24. The process of claim 19, wherein the reaction mixture is heated to a temperature ranging from about 80° C. to about 140° C.

25. The process of claim 19, wherein the polyol ester is selected from the group consisting of a monoester, a diester, a triester, and a multiester.

26. The process of claim 24, wherein the molar ratio of polyol to carboxylic acid compound ranges from about 0.01:1 to about 50:1.

27. The process of claim 24, wherein the molar ratio of polyol and carboxylic acid compound to triflate-substituted catalyst ranges from about 300:1 to about 5000:1.

28. A feed composition comprising a compound of claim 1.

29. The feed composition of claim 28, wherein the composition is formulated for a subject selected from the group consisting of a companion animal, a ruminant animal, a monogastric animal, poultry, and an aquatic animal.

30. The composition of claim 28, wherein the feed composition is selected from the group consisting of a dry supplement, and an aqueous formulation.
